(19) United States
(12) Patent Application Publication
(10) Pub. No.: US 2006/0068998 A1
(43) Pub. Date: Mar. 30, 2006

(54) Lubricant composition and triazine ring-containing compound

(75) Inventors: Masayuki Negoro, Minami-ashigara-shi (JP); Ken Kawata, Minami-ashigara-shi (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Fuji Photo Film Co. Ltd., Minami-ashigara-shi (JP)

(21) Appl. No.: 11/235,159

(22) Filed: Sep. 27, 2005

(30) Foreign Application Priority Data
Sep. 27, 2004 (JP) 2004-279100

(51) Int. Cl. C10M 133/42; C07D 251/00

(52) U.S. Cl. 508/257; 508/258; 544/180; 544/196

(57) ABSTRACT

A lubricant composition comprising a compound represented by the following formula that can maintain a low friction and a high abrasion resistance on the sliding surface over an extended period of time: 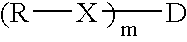
wherein D represents a cyclic group; X's each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination; R's each independently represent an alkyl group, an alkenyl group, an alkynyl group, an aryl group or a heterocyclic group; m represents an integer of from 2 to 11; and at least one of m groups of R's contains a partly fluorinated hydrocarbon group or a fluorinated carbon group.

### BACKGROUND OF THE INVENTION

1. Field of the Invention

The present invention belongs to the technical field of a lubricant composition to be supplied into mechanical frictional sliding portion, etc. and a triazine ring-containing compound to be incorporated therein and more particularly to the technical field of a lubricant composition which exhibits a low friction and a high abrasion resistance under an extreme pressure and an excellent durability of these properties and a triazine ring-containing compound to be incorporated therein.

2. Description of the Related Art

Lubricants are required to exhibit a reduced friction coefficient on the mechanical frictional sliding portion and maintain its effect as long as possible within a wide temperature range and a wide pressure range. The lubricants are also required to not only exert an effect of enhancing slipperiness between the frictional sliding members but also accordingly render the frictional sliding member itself resistant to abrasion. The reduction of the friction coefficient of lubricants such as engine oil on the frictional sliding portion and the prolongation of the life of the lubricants lead directly to the enhancement of fuel economy for machine driving, i.e., energy saving. The prolongation of the life of engine oil allows not only the reduction of the amount of waste oil but also the reduction of the discharged amount of CO₂ and thus is advantageous also in adaptability to atmosphere, which has been recently noticed. Further, among sliding portions in industrial machine system, bearings and gears which make sliding movement under severe frictional conditions undergo oil film breaking or seizing with a related art lubricant or grease when operated under severe lubricating conditions. The resulting abrasion scratch occasionally makes it impossible to obtain the desired low friction coefficient. As a result, the reliability of the machine can be impaired. In particular, when the size of the machine is reduced, the sliding portion must be operated under severer frictional conditions. Accordingly, the size of the machine can be difficultly reduced. It has thus been desired to provide a energy-saving lubricant which causes no abrasion or seizing even under severe conditions to enhance the reliability of machines and contribute to the reduction of the size thereof.

As a lubricant there has heretofore been normally used one comprising a lubricant base oil incorporated therein as a main component and lubricating aids such as organic compound incorporated therein. In recent years, organic molybdenum compounds have been noted as lubricating aids. These organic molybdenum compounds have been noted as material which can still exhibit excellent lubricating properties such as high abrasion resistance, high extreme pressure properties (high load resistance) and low friction even when the sliding portion of the machine moves under severe frictional conditions such as high temperature, high or low speed, high load and reduced size and weight and can effectively exhibit excellent lubricating properties under a pressure higher than that of ordinary pressure fluid lubricating conditions, i.e., under boundary lubricating conditions.

Although these organic molybdenum compounds are excellent materials which exhibit an excellent lubricating effect even under violent frictional conditions, these lubricants contain heavy metals such as molybdenum and zinc, sulfides which can be easily oxidized to produce sulfur oxides that have adverse effects on not only lubricants but also on sliding members themselves and atmosphere and phosphoric acid, which enriches the rivers and sea, in considerable amounts and thus are apparently disadvantageous in adaptability to atmosphere. Further, the molybdenum oxide/sulfide film formed on the sliding surface is gradually scraped off by friction to form a new film. Accordingly, when either the organic molybdenum compound or the organic zinc compound, which is the source of the film, runs short, the lubricating effect is suddenly lost. However, when the amount of the organic molybdenum compound and the organic zinc compound is raised, the amount of by-products produced by the scraping of the film in the system increases, giving adverse effects on the sliding machine itself. Accordingly, the rise in the amount of these organic compounds is not effective. In a system utilizing the organic molybdenum compound, the effect of enhancing fuel economy by the prolongation of life of lubricant cannot be expected too much. Thus, no related art lubricants have been provided which are free of environmental harmful materials and environmental polluting materials such as heavy metal element, phosphoric acid compound and sulfide and still can exhibit excellent lubricant properties continuously over an extended period of time.

It has been known that lubricant compositions mainly composed of a compound having a triazine structure are excellent in adaptability to atmosphere and enhancement of fuel economy due to prolongation of life of lubricant and thus exhibit lubricating properties useful as friction coefficient reducer, extreme pressure agent or abrasion inhibitor (see JP-A-2002-69472, [0006]-[0045]).

The lubricants have a variety of requirements. In recent years, with the enhancement of performance of various machines and the trend for severer working conditions, the lubricants have had severer requirements.

### SUMMARY OF THE INVENTION

The invention has been worked out in the aforementioned problems. An aim of the invention is to provide a novel compound which exhibits excellent lubricating properties not only when used in admixture with a related lubricant base oil but also when used free of lubricant base oil. Another aim of the invention is to provide a lubricant composition which can maintain a low friction and a high abrasion resistance on the sliding surface over an extended period of time particularly even under extreme pressures. A further aim of the invention is to provide a lubricant composition which is free of heavy metal elements, phosphoric acid groups and sulfides, which have a poor adaptability to atmosphere, to attain both a prolonged life and a good adaptability to atmosphere.

The inventors made extensive studies of solution to the aforementioned problems with the related art. As a result, it was found that a compound having a specific functional moiety structure exhibits excellent lubricating properties. The invention has thus been worked out on the basis of this knowledge.

In other words, the means for solving the aforementioned problems are as follows.
[1] A lubricant composition comprising a compound represented by the following formula (1): 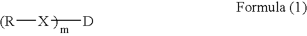
   wherein D represents a cyclic group which can bond to m groups of R—X—'s (side moieties); X's each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination; R's each independently represent an alkyl group, an alkenyl group, an alkynyl group, an aryl group or a heterocyclic group; at least one of m groups of R's contains a partly fluorinated hydrocarbon group or a fluorinated carbon group; and m represents an integer of from 2 to 11.
[1-1] The lubricant composition as defined in Clause [1], wherein R contains an aromatic group or a heterocyclic group.
[2] The lubricant composition as defined in Clause [1], wherein D is a 5- to 7-membered heterocyclic residue.
[3] The lubricant composition as defined in Clause [2], wherein the formula (1) is represented by the following formula (2): 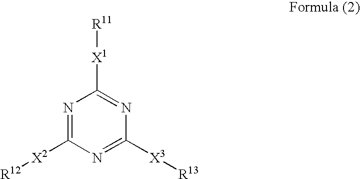
   wherein X¹, X² and X³ each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination; and R¹¹, R¹² and R¹³ each independently represent a substituted or unsubstituted alkyl, alkenyl, alkynyl, aryl or heterocyclic group, with the proviso that at least one of R¹¹, R¹² and R¹³ contains a partly fluorinated hydrocarbon group or a fluorinated carbon group.
[3-1] The lubricant composition as defined in Clause [3], wherein R¹¹ to R¹³ each contain an aromatic group or a heterocyclic group.
[4] The lubricant composition as defined in any one of Clauses [1] to [3], wherein the formula (1) is represented by the following formula (3): 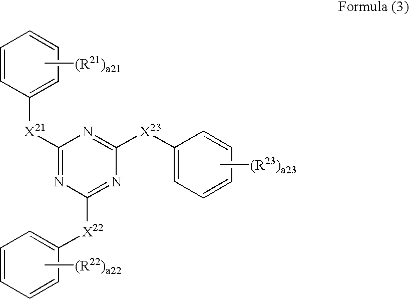
   wherein X²¹, X²² and X²³ each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination; R²¹, R²² and R²³ each independently represent a substituent, with the proviso that at least one of R²¹, R²² and R²³ contains a partly fluorinated hydrocarbon group or a fluorinated carbon group; and a21, a22 and a23 each independently represent an integer of from 1 to 5.
[4-1] The lubricant composition as defined in Clause [4], wherein R²¹ to R²³ each contain an aromatic group or a heterocyclic group.
[5] The lubricant composition as defined in any one of Clauses [1] to [4], wherein the formula (1) is represented by the following formula (4): 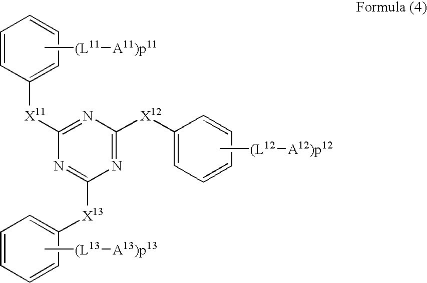
   wherein X¹¹, X¹² and X¹³ each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination; L¹¹, L¹² and L¹³ each independently represent a single bond, an NR¹ group (in which R¹ is a hydrogen atom or a C₁-C₃₀ alkyl group), an alkylene group, an oxygen atom, a sulfur atom, a carbonyl group, a sulfonyl group or a divalent linking group comprising these groups in combination; A¹¹, A¹² and A¹³ each independently represent an aromatic group or a heterocyclic group, with the proviso that at least one of A¹¹, A¹² and A¹³ contains a partly fluorinated hydrocarbon group or a fluorinated carbon group; and p¹¹, p¹² and p¹³ each independently represent an integer of from 1 to 5.
[6] The lubricant composition as defined in Clause [5], wherein all of X¹¹, X¹² and X¹³ in the formula (4) are imino group (—NH—).
[7] The lubricant composition as defined in any one of Clauses [1] to [6], wherein the formula (4) is represented by the following formula (5): 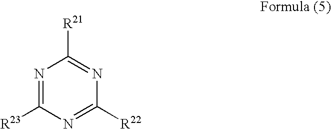
   wherein R²¹, R²² and R²³ each independently are represented by the following formula (6): 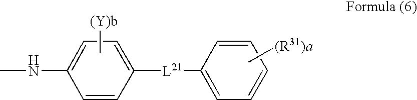
   wherein L²¹ represents a single bond, an NR¹ group (in which R¹ is a hydrogen atom or a C₁-C₃₀ alkyl group), an alkylene group, an oxygen atom, a sulfur atom, a carbonyl group, a sulfonyl group or a divalent linking group comprising these groups in combination; R³¹ represents a substituent, with the proviso that at least one of a groups of R³¹'s contains a partly fluorinated hydrocarbon group or a fluorinated carbon group; the suffix a represents 0 or an integer of from 1 to 5; Y represents a substituent; and the suffix b represents 0 or an integer of from 1 to 4.
[8] A triazine compound represented by the formula (2).
[8-1] The lubricant composition as defined in Clause [8], wherein R¹¹ to R¹³ each contain an aromatic group or a heterocyclic group.
[9] A triazine compound represented by the formula (3).
[9-1] The lubricant composition as defined in Clause [9], wherein R²¹ to R²³ each contain an aromatic group or a heterocyclic group.
[10] A triazine compound represented by the formula (4).
[11] A triazine compound represented by the formula (5).

The lubricant composition of the invention exhibits excellent abrasion resistance and extreme pressure properties and a low friction when applied to mechanical frictional sliding portion and thus has a high practicality. The lubricant composition of the invention also exhibits excellent lubricating properties even in a high temperature range and thus can maintain a low friction over an extended period of time. The compound of the invention further exhibits excellent lubricating properties not only in admixture with a lubricant base oil but also in the absence of a lubricant base oil.

### BEST MODE FOR CARRYING OUT THE INVENTION

The lubricant composition of the invention will be further described hereinafter. The description of the following constitutions will be occasionally made on the basis of representative embodiments of implementation of the invention, but the invention should not be construed as being limited thereto. The expression of numerical range “(x) to (y)” as used herein is meant to indicate the range of from numerical value (x) to numerical value (y), both inclusive as lower limit and upper limit, respectively.

The lubricant composition of the invention comprises a compound having a cyclic group and m groups of side moieties (in which m is from 2 to 11) bonding thereto. At least one of m groups of side moieties contains a partly fluorinated hydrocarbon group and a fluorinated carbon group. The term “partly fluorinated hydrocarbon group” as used herein is meant to indicate a group having fluorine atom, carbon atom and hydrogen atom. The term “fluorinated carbon group” as used herein is meant to indicate a group having fluorine atom and carbon atom but free of hydrogen atom. Since the aforementioned compound exhibits excellent lubricant properties per se, the lubricant composition of the invention may be composed of the aforementioned compound alone. The lubricant composition of the invention also may be embodied by adding the aforementioned compound to a base material such as lubricant base oil. In such an embodiment, the aforementioned compound contributes to the enhancement of the lubricating properties of the lubricant base oil when incorporated therein.

As previously mentioned, the compound to be used in the invention is a cyclic compound having a side moiety containing a partly fluorinated hydrocarbon group or a fluorinated carbon group. The term “disc-shaped compound” as used herein is meant to indicate a compound having a disc-shaped partial structure in the central part thereof. The disc-shaped partial structure is a central partial structure obtained by excluding the side moiety from the molecular structure. Explaining the morphological characteristics of the disc-shaped structure with reference to hydrogen-substituted material which is a compound that gives an original form thereof by way of example, the following expression can be made.

Firstly, the molecular size of the hydrogen-substituted material which is an original form of the disc-shaped partial structure is determined by the method comprising the following steps (1) to (5).

(1) A molecule in question is structured as planarly as possible, preferably planarly. In this case, as bond distance and bond angle there are preferably used standard values according to orbital hybridization. For details, reference can be made to “Kagaku Binran Kaitei 4-han Kisohen (Handbook of Chemistry, 4th Revised Edition, Elementary Course)”, compiled by The Chemical Society of Japan, Volume II, Chapter 15, 1993, Maruzen.

(2) The structure obtained at the step (1) is then structurally optimized as an initial value by a molecular orbital method or molecular force field method. Examples of these methods include Gaussian 92, MOPAC 93, CHARMm/QUANTA, and MM3. Preferred among these methods is Gaussian 92.

(3) The center of gravity of the structure obtained by structural optimization is moved to the origin. The coordinates fall on the principal axes of inertia (principal axes of ellipsoid of tensor of inertia).

(4) The various atoms are given a sphere defined by van der Waals force by which the shape of the molecule is depicted.

(5) The length of the various coordinate directions are measured on the van der Waals surface. These measurements are defined as a, b and c.

Defining the morphology of the disc-shaped structure by the length values a, b and c determined at the aforementioned steps (1) to (5), the morphology of the disc-shaped structure satisfies the relationships a≧b>c and a≧b≧a/2, preferably a≧b>c and a≧b≧0.7a. Alternatively, the morphology of the disc-shaped structure preferably satisfies the relationship b/2>c.

Specific examples of the disc-shaped compound as a hydrogen-substituted material that gives an original form of the disc-shaped partial structure include stereoparent compounds and derivatives thereof disclosed in “Ekisho no Kagaku (Chemistry of Liquid Crystal)”, Survey of Chemistry, Quarterly, No. 22, Chapter 5 and Section 2, Chapter 10, Compiled by The Chemical Society of Japan, issued by Japan Scientific Societies Press, 1994, C. Destrade et al, “Mol. Cryst. Liq. Cryst.”, vol. 71, page 111, 1981, B. Kohn et al, “Angew. Chem.”, vol. 96, page 70, 1984, J. M. Lehn et al, “J. Chem. Soc. Chem. Commun.”, page 1,794, 1985, and Z. Zhang, J. S. Moore et al, “J. Am. Chem. Soc.”, vol. 116, page 2,655, 1994. Examples of these disc-shaped compounds include benzene derivatives, triphenylene derivatives, trixene derivatives, phthalocyanine derivatives, porphiline derivatives, anthracene derivatives, hexaethynylbenzene derivatives, dibenzopyrene derivatives, coronene derivatives, and phenylacetylene macrocycle derivatives. Further examples of the disc-shaped compounds include electronic structures such as cyclic compounds and products of hetero atom substitution thereof disclosed in “Atarashii Houkouzoku no Kagaku (Chemistry of New Aromatic Groups)”, Survey of Chemistry, No. 15, compiled by The Chemical Society of Japan, issued by UVIVERSITY OF TOKYO PRESS, 1977.

The compound to be used as the lubricant composition of the invention is a compound represented by the following formula (1):

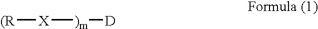

In the formula (1), D represents a cyclic group that can be bonded to m groups of side moieties. It is preferred that D be positioned at the center of the molecule and m groups of side moieties be arranged radially with D as a center. X's each independently represent a single bond, an NR¹ group (in which R¹represents a hydrogen atom or a C₁-C₃₀ alkyl group), an oxygen atom, a sulfur atom, a carbonyl group, a sulfonyl group or a divalent linking group comprising these groups in combination. R's each independently represent a substituted or unsubstituted alkyl, alkenyl, alkynyl, aryl or heterocyclic group, with the proviso that at least one of R's contains a partly fluorinated hydrocarbon group or a fluorinated carbon group. In the invention, the structure of the partly fluorinated hydrocarbon group and fluorinated carbon group are not specifically limited. The partly fluorinated hydrocarbon group and fluorinated carbon group may have a double bond, branch, cyclic structure or aromatic ring. The suffix m represents an integer of from 2 to 11.

Examples of the cyclic group represented by D in the formula (1) include aromatic groups and heterocyclic groups. Examples of the aromatic ring in the aromatic group include benzene ring, indene ring, naphthalene ring, triphenylene ring, fluorene ring, phenanthrene ring, anthracene ring, and pyrene ring. The aromatic group may contain substituents.

The heterocyclic group preferably contains a 5-, 6- or 7-membered heterocyclic ring, more preferably 5- or 6-membered ring, most preferably 6-membered ring. Preferred examples of the hetero atoms constituting the heterocyclic ring include nitrogen atom, oxygen atom, and sulfur atom. The heterocyclic ring is preferably an aromatic heterocyclic ring. The aromatic heterocyclic ring is normally an unsaturated heterocyclic ring. The aromatic heterocyclic ring is more preferably an unsaturated heterocyclic ring having most double bonds. Examples of the heterocyclic ring include furane ring, thiophene ring, pyrrole ring, pyrrolidine ring, oxazole ring, isooxazole ring, thiazole ring, isothiazole ring, imidazole ring, imidazoline ring, imidazolidine ring, pyrazole ring, pyrazoline ring, pyrazolidine ring, triazole ring, furazane ring, tetrazole ring, pyrane ring, thiine ring, pyridine ring, piperidine ring, oxazine ring, morpholine ring, thiazine ring, pyridazine ring, pyrimidine ring, pyrazine ring, piperazine ring, and triazine ring. Preferred among these heterocyclic rings is triazine ring, particularly 1,3,5-triazine ring. To these heterocyclic rings may be condensed other heterocyclic rings, aliphatic rings or aromatic rings. However, monocyclic heterocyclic rings are preferred.

In the formula (1), X represents a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination. In the case where X is a single bond, R and D may be directly bonded to each other via a nitrogen atom having a free valency as in heterocyclic group such as piperidine. Alternatively, R and D may bond to each other via a hetero atom free of free valency to form an onium salt such as oxonium salt, sulfonium salt and ammonium salt. X in the formula (1) is preferably a sulfur atom or NR¹ group in which R¹ is preferably an alkyl group having 3 or less carbon atoms or hydrogen atom.

In the formula (1), the alkyl group represented by R preferably has from 1 to 30 carbon atoms, more preferably from 2 to 30 carbon atoms, even more preferably from 4 to 30 carbon atoms, most preferably from 6 to 30 carbon atoms. The alkyl group may be straight-chain or branched. The alkyl group may have substituents. Examples of these substituents include halogen atoms, alkoxy groups (e.g., methoxy, ethoxy, methoxyethoxy, phenoxy), sulfide groups (e.g., methylthio, ethylthio, propylthio), alkylamino groups (e.g., methylamino, propylamino), acyl groups (e.g., acetyl, propanoyl, octanoyl, benzoyl), acyloxy groups (e.g., acetoxy, pivaloyloxy, benzoyloxy), hydroxyl groups, mercapto groups, amino groups, carboxyl groups, sulfo groups, carbamoyl groups, sulfamoyl groups, and ureido groups.

The preferred range of number of carbon atoms in, scope of structure of and specific examples of substituents on the alkenyl group or alkynyl group represented by R in the formula (1) are the same as described with reference to alkyl group. However, the lower limit of the number of carbon atoms is 2. The terms “alkyl group”, “alkenyl group” and “alkynyl group” as used herein are meant to include cycloalkyl group, cycloalkenyl group and cycloalkynyl group, respectively. Examples of the substituents having alkyl group, alkenyl group and alkynyl group (e.g., alkoxy group) include those having cycloalkyl group, cycloalkenyl group and cycloalkynyl group.

Examples of the aryl group represented by R in the formula (1) include phenyl group, indenyl group, α-naphthyl group, β-naphthyl group, fluorenyl group, phenanthrenyl group, anthracenyl group, and pyrenyl group. Preferred among these aryl groups are phenyl group and naphthyl group. These naphthyl groups may have substituents. Examples of these substituents include those exemplified above with reference to the substituents on the alkyl group. Other examples of these substituents include alkyl groups. Preferred examples of these substituents include those containing a straight-chain or branched alkyl residue having 8 or more carbon atoms such as alkyl group (e.g., octyl, decyl, hexadecyl, 2-ethylhexyl), alkoxy group (e.g., dodecyloxy, hexadecyloxy), sulfide group (e.g., hexadecylthio), substituted amino group (e.g., heptadecylamino), octylcarbamoyl group, octanoyl group and decylsulfamoyl group. The aryl group is preferably substituted by two or more of these substituents. The aryl group may be further substituted by halogen atoms, hydroxyl groups, cyano groups, nitro groups, carboxyl groups, sulfo groups or the like besides the aforementioned substituents.

The heterocyclic group represented by R in the formula (1) is preferably a 5- to 7-membered heterocyclic ring, more preferably a 5- or 6-membered heterocyclic ring, most preferably a 6-membered heterocyclic ring as D is. Specific examples of these skeletons include those disclosed in “Iwanami Rikagaku Jiten (Dictionary of Physics and Chemistry)”, 3rd revised and enlarged edition, Chapter 11 of supplement: Nomenclature of Organic Chemistry; Table 4: Name of Main Heteromonocyclic Compounds (page 1,606); and Table 5: Name of Main Condensed Heteromonocyclic Compounds (page 1,607). These heterocyclic groups may have substituents as the aryl group R does. These heterocyclic groups are preferably substituted by substituents containing a straight-chain or branched alkyl residue having 8 or more carbon atoms. These heterocyclic groups each are preferably substituted by two or more of these substituents. These heterocyclic groups may be further substituted by halogen atoms, hydroxyl groups, cyano groups, nitro groups, carboxyl groups, sulfo groups or the like besides the aforementioned substituents.

In the formula (1), at least one of m groups of R′s contains a partly fluorinated hydrocarbon group or a fluorinated carbon group. The suffix m represents an integer of from 2 to 11. When m is 2 or more, two or more X's and R's maybe the same or different, respectively. Preferably, the plurality of X's and R's are the same, respectively. The suffix m is preferably 3 or more.

Preferred among the compounds represented by the formula (1) are those represented by the following formula (2):

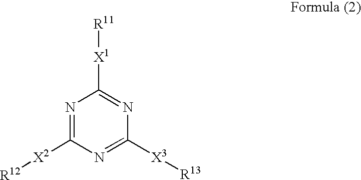

In the formula (2), X¹, X² and X³ each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination. In the case where X¹, X² and X³ each are a single bond, R¹¹, R¹² and R¹³ may directly bond to the main moiety of the molecule via a nitrogen atom having a free valency as in heterocyclic group such as piperidine. Alternatively, R¹¹, R¹² and R¹³ may bond to the main moiety of the molecule via a hetero atom free of free valency to form an onium salt such as oxonium salt, sulfonium salt and ammonium salt. In the case where X¹, X²and X³ each are not a single bond, they are each an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination. Examples of the divalent linking group comprising these groups in combination include oxycarbonyl group, ureylene group, oxysulfonyl group, —NR¹CO—, and —SO₂NR¹— (in which R¹ represents a C₁-C₃₀ alkyl group or hydrogen atom). Specific examples of the divalent linking group specify specific examples of the structure extending from the triazine ring side to the substituted phenyl group. X¹, X² and X³ each are preferably a sulfur atom or NR¹ group. R¹ is preferably an alkyl group having 3 or less carbon atoms or a hydrogen atom. X¹, X² and X³ each are more preferably an imino group (—NH—). It is preferred that X¹, X² and X³ be the same.

In the formula (2), R¹¹, R¹² and R¹³ each independently represent a substituted or unsubstituted alkyl, alkenyl, alkynyl, aryl or heterocyclic group. At least one of R¹¹, R¹² and R¹³ contains a partly fluorinated hydrocarbon group or a fluorinated carbon group.

The alkyl groups represented by R¹¹, R¹² and R¹³ each preferably have from 1 to 30 carbon atoms, more preferably from 2 to 30 carbon atoms, even more preferably from 4 to 30 carbon atoms, most preferably from 6 to 30 carbon atoms. The alkyl group may be straight-chain or branched. The alkyl group also may have substituents. Examples of these substituents include halogen atoms, alkoxy groups (e.g., methoxy, ethoxy, methoxyethoxy, phenoxy), sulfide groups (e.g., methylthio, ethylthio, propylthio), alkylamino groups (e.g., methylamino, propylamino), acyl groups (e.g., acetyl, propanoyl, octanoyl, benzoyl), acyloxy groups (e.g., acetoxy, pivaloyloxy, benzoyloxy), hydroxyl groups, mercapto groups, amino groups, carboxyl groups, sulfo groups, carbamoyl groups, sulfamoyl groups, and ureido groups. The preferred range of number of carbon atoms in, scope of structure of and specific examples of substituents on the alkenyl group or alkynyl group represented by R¹¹, R¹² and R¹³ are the same as described with reference to alkyl group. However, the lower limit of the number of carbon atoms in the alkenyl group or alkynyl group is 2.

The aryl groups represented by R¹¹, R¹² and R¹³ each preferably have from 6 to 50 carbon atoms, more preferably from 6 to 40 carbon atoms, even more preferably from 6 to 30 carbon atoms. Examples of the aryl group include phenyl group, indenyl group, α-naphthyl group, β-naphthyl group, fluorenyl group, phenanthrenyl group, anthracenyl group, and pyrenyl group. Preferred among these aryl groups are phenyl group and naphthyl group. The aryl group is preferably further substituted by substituents containing a straight-chain or branched alkyl residue having 8 or more carbon atoms such as alkyl group (e.g., octyl, decyl, hexadecyl, 2-ethylhexyl), alkoxy group (e.g., dodecyloxy, hexadecyloxy, 2-hexyldecyloxy, hexyloxyethyleneoxyethyleneoxy), sulfide group (e.g., hexadecylthio), substituted amino group (e.g., heptadecylamino), octylcarbamoyl group, arylcarbonyl group (e.g., benzoyl), arylcarbonyloxy group (e.g., benzoyloxy), alkanoyl group (e.g., octanoyl) and decylsulfamoyl group. More preferably, these substituents contain a partly fluorinated hydrocarbon group or a fluorinated carbon group. The aryl group may be further substituted by halogen atoms, hydroxyl groups, cyano groups, nitro groups, carboxyl groups, sulfo groups or the like besides the aforementioned substituents.

The heterocyclic groups represented by R¹¹, R¹² and R¹³ each preferably have from 3 to 50 carbon atoms, more preferably from 3 to 40 carbon atoms, even more preferably from 3 to 30 carbon atoms. The heterocyclic group is preferably a 5- to 7-membered heterocyclic ring, more preferably a 5- or 6-membered heterocyclic ring, most preferably a 6-membered heterocyclic ring as D in the formula (1) is. Specific examples of these skeletons include those disclosed in “Iwanami Rikagaku Jiten (Dictionary of Physics and Chemistry)”, 3rd revised and enlarged edition, Chapter 11 of supplement: Nomenclature of Organic Chemistry; Table 4: Name of Main Heteromonocyclic Compounds (page 1,606); and Table 5: Name of Main Condensed Heteromonocyclic Compounds (page 1,607). These heterocyclic groups are preferably substituted by substituents containing a straight-chain or branched alkyl residue having 8 or more carbon atoms as the aryl group is. More preferably, these substituents each contain a partly fluorinated hydrocarbon group or a fluorinated carbon group. These heterocyclic groups may be further substituted by halogen atoms, hydroxyl groups, cyano groups, nitro groups, carboxyl groups, sulfo groups or the like besides the aforementioned substituents.

It is preferred that R¹¹, R¹² and R¹³ all contain a partly fluorinated hydrocarbon group or a fluorinated carbon group. R¹¹, R¹² and R¹³ are preferably the same.

More desirable embodiments of the compound represented by the formula (2) include a compound represented by the following formula (3): 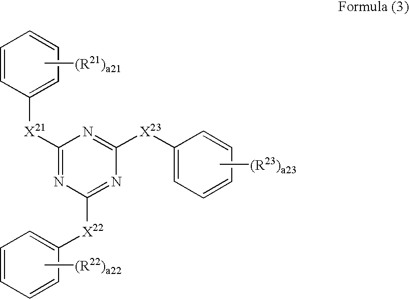
wherein X²¹, X²² and X²³ each independently represent a single bond, an NR¹ group (in which R¹ represents a hydrogen atom or C₁-C₃₀ alkyl group), an oxygen atom, a sulfur atom, a carbonyl group, a sulfonyl group or a divalent linking group comprising these groups in combination; R²¹, R²² and R²³ each independently represent a substituent, with the proviso that at least one of R²¹, R²² and R²³ contains a partly fluorinated hydrocarbon group or a fluorinated carbon group; and a21, a22 and a23 each independently represent an integer of from 1 to 5.

In the formula (3), X²¹, X²² and X²³ each independently represent a single bond, an NR¹ group (in which R¹ represents a hydrogen atom or C₁-C₃₀ alkyl group), an oxygen atom, a sulfur atom, a carbonyl group, a sulfonyl group or a divalent linking group comprising these groups in combination. In the case where X²¹, X²² and X²³ each are not a single bond, X²¹, X²² and X²³ each are an NR¹ group (in which R¹ represents a hydrogen atom or C₁-C₃₀ alkyl group), an oxygen atom, a sulfur atom, a carbonyl group, a sulfonyl group or a divalent linking group comprising these groups in combination. Examples of the divalent linking group comprising these groups in combination include oxycarbonyl group, ureylene group, oxysulfonyl group, —NR¹CO—, and —SO₂NR¹— (in which R¹ represents a C₁-C₃₀ alkyl group or hydrogen atom). Specific examples of the divalent linking group specify specific examples of the structure extending from the triazine ring side to the substituted phenyl group. X²¹, X²² and X²³ each are preferably a sulfur atom or NR¹ group. R¹ is preferably an alkyl group having 3 or less carbon atoms or a hydrogen atom. X²¹, X²² and X²³ each are more preferably an imino group (—NH—) It is preferred that X²¹, X²² and X²³ be the same.

Examples of substituents X²¹, X²² and X²³ include halogen atoms (e.g., chlorine atom, bromine atom, iodine atom), alkyl groups [straight-chain, branched or cyclic substituted or unsubstituted alkyl group, including alkyl groups (preferably C₁-C₃₀ alkyl group such as methyl, ethyl, n-propyl, isopropyl, t-butyl, n-octyl, eicosil, 2-chloroethyl, 2-cyanoethyl, 2-ethylhexyl), cycloalkyl groups (preferably C₃-C₃₀ substituted or unsubstituted cycloalkyl group such as cyclohexyl, cyclopentyl and 4-n-dodecylcyclohexyl), bicyclohexyl groups (preferably C₅-C₃₀ substituted or unsubstituted bicycloalkyl group, i.e., monovalent group obtained by removing one hydrogen atom from C₅-C₃₀ bicycloalkane, e.g., bicyclo[1,2,2]heptane-2-il, bicyclo[2,2,2]octane-3-il and tricyclo structure having many cyclic structures. The alkyl groups (e.g., alkyl group in alkylthio group) in the following substituents, too, represent an alkyl group having such a concept.], alkenyl groups [straight-chain, branched or cyclic substituted or unsubstituted alkenyl group, including alkenyl groups (preferably C₂-C₃₀ substituted or unsubstituted alkenyl group such as vinyl, allyl, prenyl, geranyl, oleyl), cycloalkenyl groups (preferably C₃-C₃₀ substituted or unsubstituted cycloalkenyl group, i.e., monovalent group obtained by removing one hydrogen atom from C₃-C₃₀ cycloalkene such as 2-cyclopentene-1-il and 2-cyclohexene-1-il), bicycloalkenyl groups (substituted or unsubstituted bicycloalkenyl group, preferably C₅-C₃₀ substituted or unsubstituted bicycloalkenyl group, i.e., monovalent group obtained by removing one hydrogen atom from bicycloalkene having one double bond, e.g., bicyclo[2,2,1]hepto-2-ene-1-il, bicyclo[2,2,2]octo-2-ene-4-il], alkynyl groups (preferably C₂-C₃₀ substituted or unsubstituted alkynyl group such as ethinyl, propargyl and trimethylsiilylethinyl), aryl groups (preferably C₆-C₃₀ substituted or unsubstituted aryl group such as phenyl, p-tolyl, naphthyl, m-chlorophenyl and o-hexadecanoylaminophenyl), heterocyclic groups (preferably monovalent group obtained by removing one hydrogen atom from a 5- or 6-membered substituted or unsubstituted aromatic or nonaromatic heterocyclic compound, more preferably a C₃-C₃₀ 5- or 6-membered aromatic heterocyclic group such as 2-furyl, 2-chenyl, 2-pyrimidinyl and 2-benzothiazolyl), cyano groups, hydroxyl groups, nitro groups, carboxyl groups, alkoxy groups (preferably C₁-C₃₀ substituted or unsubstituted alkoxy group such as methoxy, ethoxy, isopropoxy, t-butoxy, n-octyloxy and 2-methoxyethoxy), aryloxy groups (preferably C₆-C₃₀ substituted or unsubstituted aryloxy group such as phenoxy, 2-methylphenoxy, 4-t-butylphenoxy, 3-nitrophenoxy and 2-tetradecanoylamino phenoxy), silyloxy groups (preferably C₃-C₂₀ silyloxy group such as trimethylsilyloxy and t-butyldimethyl silyloxy), heterocyclic oxy groups (preferably C₂-C₃₀ substituted or unsubstituted heterocyclic oxy group such as 1-phenyltetrazole-5-oxy and 2-tetrahydropyranyloxy), acyloxy groups (preferably formyloxy group, C₂-C₃₀ substituted or unsubstituted alkylcarbonyloxy group or C₆-C₃₀ substituted or unsubstituted arylcarbonyloxy group such as formyloxy, acetyloxy, pivaloyloxy, stearoyloxy, benzoyloxy and p-methoxyphenylcarbonyloxy), carbamoyloxy groups (preferably C₁-C₃₀ substituted or unsubstituted carbamoyloxy group such as N,N-dimethylcarbamoyloxy, N,N-diethylcarbamoyloxy, morpholinocarbonyloxy, N,N-di-n-octylaminocarbonyloxy and N-n-octylcarbamoyloxy), alkoxycarbonyloxy groups (preferably C₂-C₃₀ substituted or unsubstituted alkoxycarbonyloxy group such as methoxycarbonyloxy, ethoxycarbonyloxy, t-butoxycarbonyloxy and n-octylcarbonyloxy), aryloxycarbonyloxy groups (preferably C₇-C₃₀ substituted or unsubstituted aryloxy carbonyloxy group such as phenoxycarbonyloxy, p-methoxyphenoxycarbonyloxy and p-n-hexadecyloxyphenoxy carbonyloxy), amino groups (preferably amino group, C₁-C₃₀ substituted or unsubstituted alkylamino group or C₆-C₃₀ substituted or unsubstituted anilino group such as amino, methylamino, dimethylamino, anilino, N-methyl-anilino and diphenylamino), acylamino groups (preferably formylamino group, C₁-C₃₀ substituted or unsubstituted alkylcarbonylamino group or C₆-C₃₀ substituted or unsubstituted arylcarbonylamino group such as formylamino, acetylamino, pivaloylamino, lauroylamino, benzoylamino and 3,4,5-tri-n-octyloxy phenylcarbonylamino), aminocarbonylamino groups (preferably C₁-C₃₀ substituted or unsubstituted aminocarbonylamino group such as carbamoylamino, N,N-dimethylaminocarbonylamino, N,N-diethylaminocarbonyl amino and morpholinocarbonylamino), alkoxycarbonylamino groups (preferably C₂-C₃₀ substituted or unsubstituted alkoxycarbonylamino group such as methoxycarbonylamino, ethoxycarbonylamino, t-butoxycarbonylamino, n-octadecyloxycarbonylamino and N-methyl-methoxycarbonylamino), aryloxycarbonylamino groups (preferably C₇-C₃₀ substituted or unsubstituted aryloxycarbonylamino group such as phenoxycarbonylamino, p-chlorophenoxycarbonylamino and m-n-octyloxyphenoxy carbonylamino), sulfamoylamino groups (preferably C₀-C₃₀ substituted or unsubstituted sulfamoylamino group such as sulfamoylamino, N,N-dimethylaminosulfonylamino and N-n-octylaminosulfonylamino), alkylsulfonylamino groups and arylsulfonylamino groups (preferably C₁-C₃₀ substituted or unsubstituted alkylsulfonylamino group or C₆-C₃₀ substituted or unsubstituted arylsulfonylamino group such as methylsulfonylamino, butylsulfonylamino, phenylsulfonylamino, 2,3,5-trichlorophenylsulfonyl amino and p-methylphenylsulfonylamino), mercapto groups, alkylthio groups (preferably C₁-C₃₀ substituted or unsubstituted alkylthio group such as methylthio, ethylthio and n-hexadecylthio), arylthio groups (preferably C₆-C₃₀ substituted or unsubstituted arylthio group such as phenylthio, p-chlorophenylthio, m-methoxy phenylthio, 2-butoxy-5-t-butylphenylthio, 4-hexanoyl aminophenylthio and 2-benzamidephenylthio), heterocyclic thio groups (preferably C₂-C₃₀ substituted or unsubstituted heterocyclic thio group such as 2-benzothiazolylthio, 1-phenyltetrazole-5-ilthio and 1,3,4-thiadiazole-2-ilthio), sulfamoyl groups (preferably C₀-C₃₀ substituted or unsubstituted sulfamoyl group such as N-ethylsulfamoyl, N-(3-dodecyloxypropyl)sulfamoyl, N,N-dimethylsulfamoyl, N-acetylsulfamoyl, N-benzoylsulfamoyl, N-(N′-phenylcarbamoyl)sulfamoyl), sulfo groups, alkylsulfinyl groups and arylsulfinyl groups (preferably C₁-C₃₀ substituted or unsubstituted alkylsulfinyl group or C₆-C₃₀ arylsulfinyl group such as methyl sulfinyl, ethyl sulfinyl, phenyl sulfinyl and p-methylphenyl sulfinyl), alkylsulfonyl groups and arylsulfonyl groups (preferably C₁-C₃₀ substituted or unsubstituted alkylsulfonyl group or C₆-C₃₀ substituted or unsubstituted arylsulfonyl group such as methyl sulfonyl, ethyl sulfonyl, phenyl sulfonyl and p-methylphenyl sulfonyl), acyl groups (preferably formyl group, C₂-C₃₀ substituted or unsubstituted alkylcarbonyl group or C₇-C₃₀ substituted or unsubstituted arylcarbonyl group such as acetyl, pivaloyl, 2-chloroacetyl, stearoyl, benzoyl and p-n-octyloxyphenylcarbonyl), aryloxycarbonyl groups (preferably C₇-C₃₀ substituted or unsubstituted aryloxycarbonyl group such as phenoxycarbonyl, o-chlorophenoxycarbonyl, m-nitrophenoxycarbonyl and p-t-butylphenoxycarbonyl), alkoxycarbonyl groups (preferably C₂-C₃₀ substituted or unsubstituted alkoxycarbonyl group such as methoxycarbonyl, ethoxycarbonyl, t-butoxycarbonyl and n-octadecyloxycarbonyl), carbamoyl groups (preferably C₁-C₃₀ substituted or unsubstituted carbamoyl group such as carbamoyl, N-methylcarbamoyl, N,N-dimethylcarbamoyl, N,N-di-n-octylcarbamoyl and N-(methylsulfonyl)carbamoyl), arylazo groups and heterocyclic azo groups (preferably C₆-C₃₀ substituted or unsubstituted arylazo group and C₃-C₃₀ substituted or unsubstituted heterocyclic azo group such as phenylazo, p-chlorophenyl azo and 5-ethylthio-1,3,4-thiadiazole-2-ilazo), imide groups (preferably N-succinimide, N-phthalimide), phosphino groups (preferably C₂-C₃₀ substituted or unsubstituted phosphino group such as dimethyl phosphino, diphenyl phosphino and methyl phenoxy phosphino), phosphinyl groups (preferably C₂-C₃₀ substituted or unsubstituted phosphinyl group such as phosphinyl, dioctyloxy phosphinyl and diethoxy phosphinyl), phosphinyloxy groups (preferably C₂-C₃₀ substituted or unsubstituted phosphinyloxy groups such as diphenoxy phosphinyloxy and dioctyloxy phosphinyloxy), phosphinylamino groups (preferably C₂-C₃₀ substituted or unsubstituted phosphinylamino group such as dimethoxyphosphinylamino and dimethylaminophosphinyl amino), and silyl groups (preferably C₃-C₃₀ substituted or unsubstituted silyl group such as trimethylsilyl, t-butyldimethylsilyl and phenyldimethylsilyl).

Further examples of the substituents R²¹, R²² and R²³ include those obtained by substituting these substituents by one or more selected from the group consisting of these substituents. At least one of R²¹, R²² and R²³ contains a partly fluorinated hydrocarbon group or a fluorinated carbon group. Further, at least one of R²¹, R²² and R²³ is preferably an acyl group (e.g., arylcarbonyl, alkylcarbonyl), arylcarbonyloxy group, aryloxy group or alkoxy group substituted by a substituent containing a straight-chain or branched alkyl residue containing a partly fluorinated hydrocarbon group or a fluorinated carbon group.

The suffixes a²¹, a²² and a²³ each independently represent an integer of from 1 to 5, preferably from 1 to 3. When a²¹, a²² and a²³ each are 2 or more, the two or more R²¹'s, R²²'s and R²³'s may be the same or different, respectively. Preferably, R²¹'s, R²²'s and R²³'s are the same, respectively.

A more desirable embodiment of the compound represented by the formula (3) is a compound represented by the following formula (4): 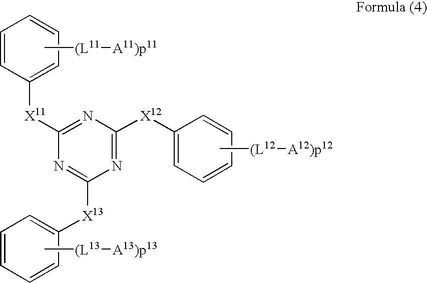
wherein X¹¹, X¹² and X¹³ each independently represent a single bond, an NR¹ group (in which R¹ represents a hydrogen atom or a C₁-C₃₀ alkyl group), an oxygen atom, a sulfur atom, a carbonyl group, a sulfonyl group or a divalent linking group comprising these groups in combination; L¹¹, L¹² and L¹³ each independently represent a single bond, an NR¹ group (in which R¹ is a hydrogen atom or a C₁-C₃₀ alkyl group), an alkylene group, an oxygen atom, a sulfur atom, a carbonyl group, a sulfonyl group or a divalent linking group comprising these groups in combination; A¹¹, A¹² and A¹³ each independently represent an aromatic group or heterocyclic group, with the proviso that at least one of A¹¹, A¹² and A¹³ contains a partly fluorinated hydrocarbon group or fluorinated carbon group; and p¹¹, p¹² and p¹³ each independently represent an integer of from 1 to 5.

Examples of the divalent linking group comprising these groups in combination represented by X¹¹, X¹² and X¹³ include oxycarbonyl group, ureylene group, oxysulfonyl group, —NR¹CO—, and —SO₂NR¹— (in which R¹ represents a C₁-C₃₀ alkyl group or hydrogen atom). Specific examples of the divalent linking group specify specific examples of the structure extending from the triazine ring side to the substituted phenyl group. X¹¹, X¹² and X¹³ each are preferably a sulfur atom an oxygen atom or an NR¹ group. R¹ is preferably an alkyl group having 3 or less carbon atoms or a hydrogen atom. X¹¹, X¹² and X¹³ each are more preferably an imino group (—NH—). It is preferred that X¹¹, X¹² and X¹³ be the same.

L¹¹, L¹² and L¹³ each independently represent a single bond, an NR¹ group (in which R¹ is a hydrogen atom or a C₁-C₃₀ alkyl group), an alkylene group, an oxygen atom, a sulfur atom, a carbonyl group, a sulfonyl group or a divalent linking group comprising these groups in combination, preferably an oxygen atom, an oxyalkylene group, an oxycarbonyl group, a carbonyloxy group, a carbonyl group, and —NR¹CO— (in which R¹ is a hydrogen atom or a C₁-C₃₀ alkyl group), and more preferably an oxycarbonyl group, a carbonyloxy group and a carbonyl group. L¹¹, L¹² and L¹³ are preferably the same.

A¹¹, A¹² and A¹³ each independently represent an aromatic group or heterocyclic group. Examples of the aromatic ring in the aromatic group include benzene ring, indene ring, naphthalene ring, triphenylene ring, fluorene ring, phenanthrene ring, anthracene ring, and pyrene ring. Preferred among these aromatic rings are benzene ring and naphthalene ring.

The heterocyclic group preferably has a 5-, 6- or 7-membered heterocyclic ring, more preferably 5- or 6-membered heterocyclic ring. Preferred examples of the hetero atoms constituting the heterocyclic ring include nitrogen atom, oxygen atom, and sulfur atom. The heterocyclic ring is preferably an aromatic heterocyclic ring. The aromatic heterocyclic ring is normally an unsaturated heterocyclic ring. The aromatic heterocyclic ring is more preferably an unsaturated heterocyclic ring having most double bonds. Examples of the heterocyclic ring include furane ring, thiophene ring, pyrrole ring, pyrrolidine ring, oxazole ring, isooxazole ring, thiazole ring, isothiazole ring, imidazole ring, imidazoline ring, imidazolidine ring, pyrazole ring, pyrazoline ring, pyrazolidine ring, triazole ring, furazane ring, tetrazole ring, pyrane ring, thiine ring, pyridine ring, piperidine ring, oxazine ring, morpholine ring, thiazine ring, pyridazine ring, pyrimidine ring, pyrazine ring, piperazine ring, and triazine ring. To these heterocyclic rings may be condensed other heterocyclic rings, aliphatic rings or aromatic rings. However, monocyclic heterocyclic rings are preferred. These heterocyclic rings may have substituents as the aromatic group does.

At least one of A¹¹, A¹² and A¹³ is substituted by a substituent containing a partly fluorinated hydrocarbon group or a fluorinated carbon group. More preferably, at least one of A¹¹, A¹² and A¹³ is substituted by an acyl group (e.g., arylcarbonyl, alkylcarbonyl), arylcarbonyloxy group, aryloxy group or alkoxy group substituted by a substituent containing a straight-chain or branched alkyl residue containing a partly fluorinated hydrocarbon group or a fluorinated carbon group.

The suffixes p¹¹, p¹² and p¹³ each independently represent an integer of from 1 to 5, preferably from 1 to 3. When p¹¹, p¹² and p¹³ each are 2 or more, the two or more (L¹¹-A¹¹)'s, (L¹²-A¹²)'s and (L¹³-A¹³)'s may be the same or different, respectively. Preferably, (L¹¹-A¹¹)'s, (L¹²-A¹²)'s and (L¹³-A¹³)'s are the same, respectively.

A¹¹, A¹² and A¹³ are preferably the same.

Preferred among the compounds represented by the formula (4) is one represented by the following formula (5): 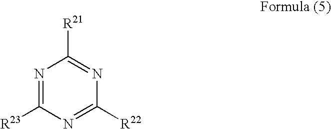
wherein R²¹, R²² and R²³ each independently are represented by the following formula (6). R²¹, R²² and R²³ are preferably the same.

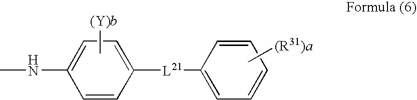

In the formula (6), L²¹ represents a single bond, an NR¹ group (in which R¹ is a hydrogen atom or a C₁-C₃₀ alkyl group), an alkylene group, an oxygen atom, a sulfur atom, a carbonyl group, a sulfonyl group or a divalent linking group comprising these groups in combination. Preferred examples of the divalent linking group comprising these groups in combination include oxygen atom, oxyalkylene group, oxycarbonyl group, carbonyloxy group, carbonyl group, and —NR¹CO— (in which R¹ represents a C₁-C₃₀ alkyl group or hydrogen atom) . More desirable among these groups are oxycarbonyl group and carbonyl group.

R³¹ and Y each represent a substituent. Examples of the substituents R³¹ and Y include those listed with reference to the substituents R²¹, R²² and R²³ in the formula (3). Further examples of the substituents R³¹ and Y include those obtained by substituting these substituents by one or more substituents selected from the group consisting of these substituents.

At least one of a groups of R³¹'s contains a partly fluorinated hydrocarbon group or a fluorinated carbon group. Preferred examples of the substituent R³¹ include alkoxy group, alkoxycarbonyl group and acyl group substituted by a substituent containing a straight-chain or branched alkyl residue containing a partly fluorinated hydrocarbon group or a fluorinated carbon group. The number of carbon atoms in R³¹ is preferably from 1 to 40, more preferably from 1 to 20. The suffix a represents an integer of from 1 to 5, preferably from 1 to 3. When the suffix a is 2 or more, the two or more R³¹'s may be the same or different. Preferably, the two or more R³¹'s are the same.

Preferred examples of the substituent Y include halogen atom, alkyl group, and alkoxy group. Preferred examples of the halogen atom include fluorine atom, chlorine atom, and bromine atom. More desirable among these halogen atoms is chlorine atom. The number of carbon atoms in Y is preferably from 1 to 20, more preferably from 1 to 10. The suffix b represents 0 or an integer of from 1 to 4, preferably 0 or 1, more preferably 0. When the suffix b is 2 or more, the two or more Y's may be the same or different. Preferably, the two or more Y's are the same.

R²¹, R²² and R²³ in the compound represented by the formula (5) each independently are one represented by the following formula (7): 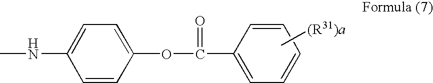
wherein R³¹ represents a substituent; and the suffix a represents 0 or an integer of from 1 to 5. Specific examples of R³¹ and the suffix a are the same as the specific and preferred examples of R³¹ and the suffix a in the formula (6).

Also preferably, R²¹, R²² and R²³ in the compound represented by the formula (5) each preferably are independently represented by the following formula (8): 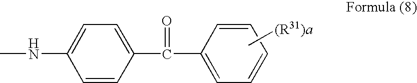
wherein R³¹ represents a substituent; and the suffix a represents 0 or an integer of from 1 to 5. Specific examples of R³¹ and the suffix a are the same as the specific and preferred examples of R³¹ and the suffix a in the formula (6).

Specific examples of the compound represented by the formula (1) will be given below with the structure of the formula (2) as a representative example, but the compound to be incorporated in the lubricant composition of the invention is not limited thereto.

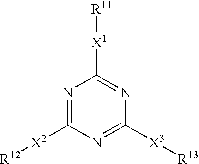

(—X¹—R¹¹)═(—X²—R¹²)═(—X³—R¹³)

| | |
|---|---|
| A-1 | 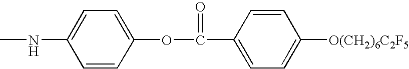 |
| A-2 | 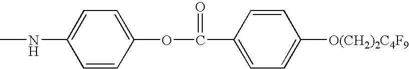 |
| A-3 | 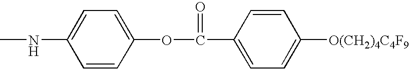 |
| A-4 | 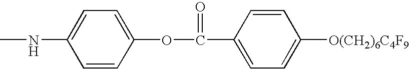 |
| A-5 | 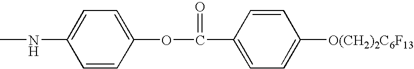 |
| A-6 | 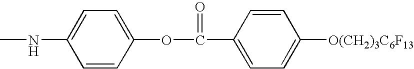 |
| A-7 | 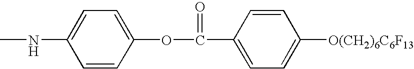 |
| A-8 | 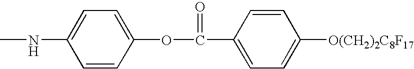 |
| A-9 | 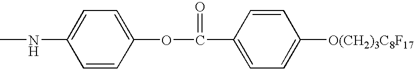 |
| A-10 | 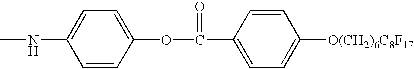 |
| A-11 | 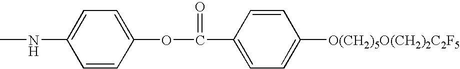 |
| A-12 | 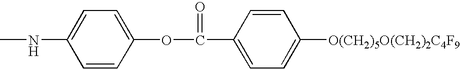 |
| A-13 | 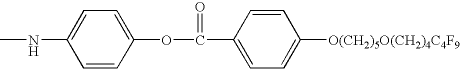 |
| A-14 | 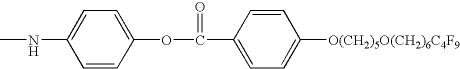 |
| A-15 | 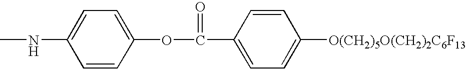 |
| A-16 | 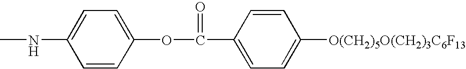 |
| A-17 | 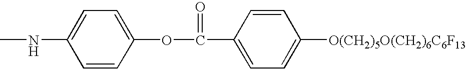 |
| A-18 | 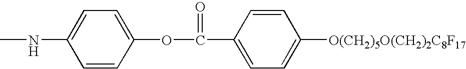 |
| A-19 | 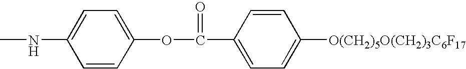 |
| A-20 | 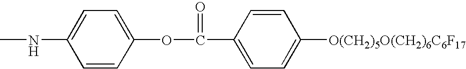 |
| A-21 | 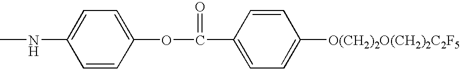 |
| A-22 | 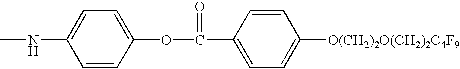 |
| A-23 | 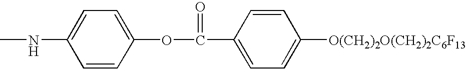 |
| A-24 | 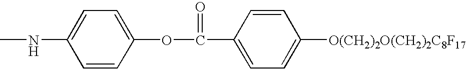 |
| A-25 | 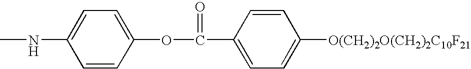 |
| B-1 | 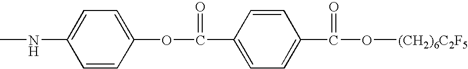 |
| B-2 | 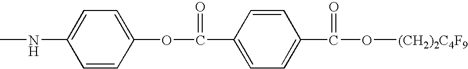 |
| B-3 | 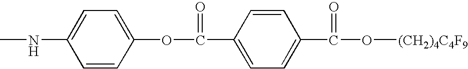 |
| B-4 | 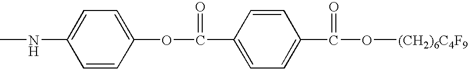 |
| B-5 | 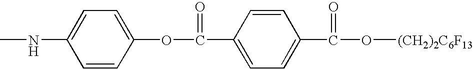 |
| B-6 | 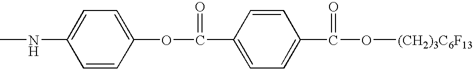 |
| B-7 | 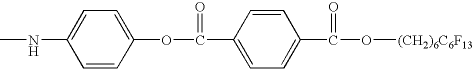 |
| B-8 | 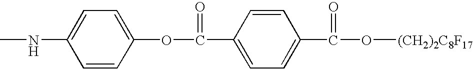 |
| B-9 | 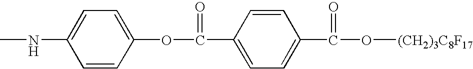 |
| B-10 | 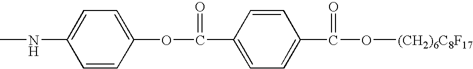 |
| B-11 | 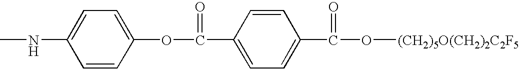 |
| B-12 | 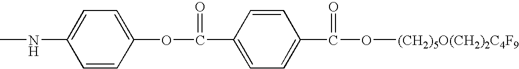 |
| B-13 | 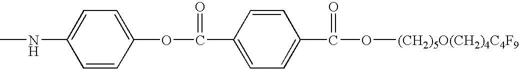 |
| B-14 | 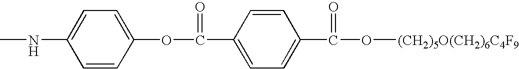 |
| B-15 | 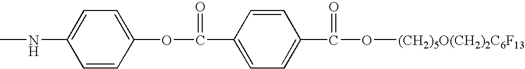 |
| B-16 | 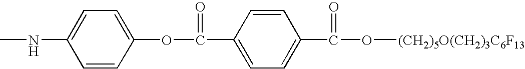 |
| B-17 | 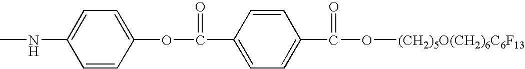 |
| B-18 | 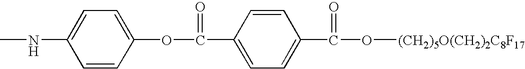 |
| B-19 | 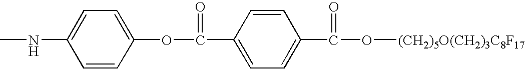 |
| B-20 | 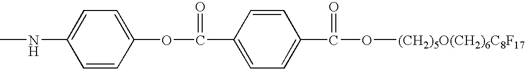 |
| B-21 | 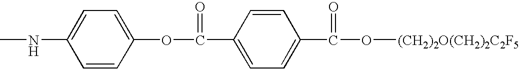 |
| B-22 | 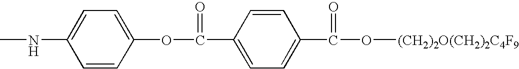 |
| B-23 | 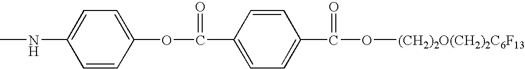 |
| B-24 | 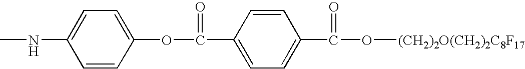 |
| B-25 | 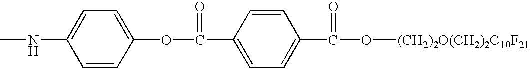 |
| B-26 | 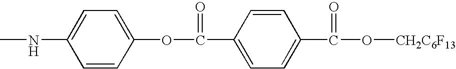 |
| B-27 | 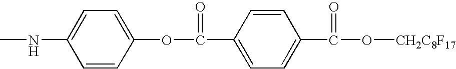 |
| B-28 | 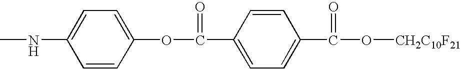 |
| B-29 | 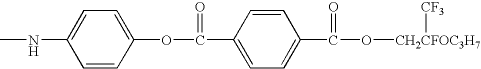 |
| B-30 | 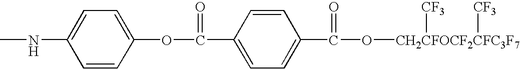 |
| B-31 | 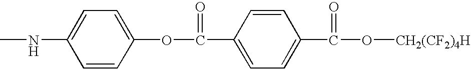 |
| B-32 | 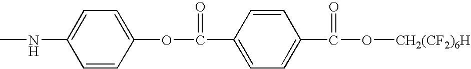 |
| B-33 | 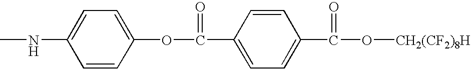 |
| B-34 | 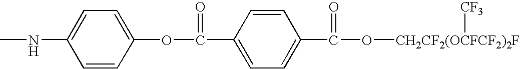 |
| B-35 | 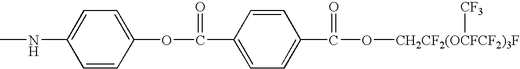 |
| B-36 | 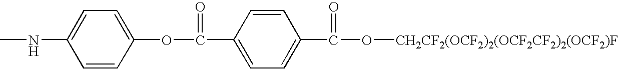 |
| B-37 | 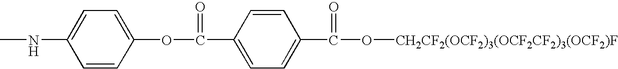 |
| B-38 | 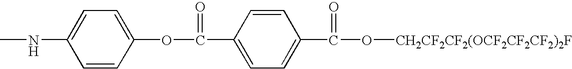 |
| B-39 | 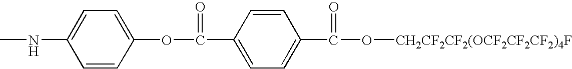 |
| B-40 | 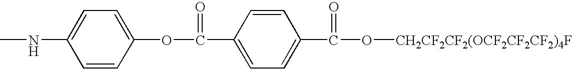 |
| C-1 | 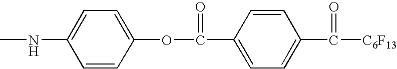 |
| C-2 | 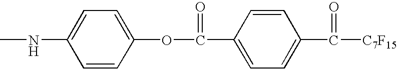 |
| C-3 | 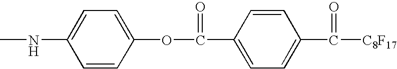 |
| C-4 | 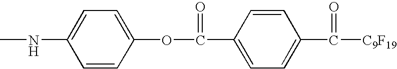 |
| C-5 | 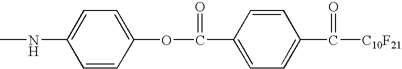 |
| C-6 | 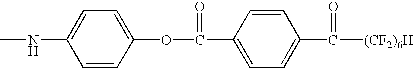 |
| C-7 | 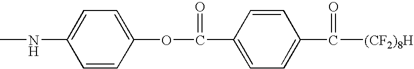 |
| C-8 | 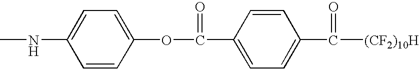 |
| C-9 | 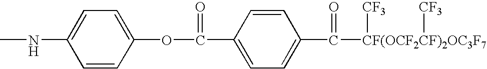 |
| C-10 | 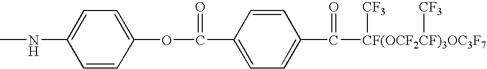 |
| C-11 | 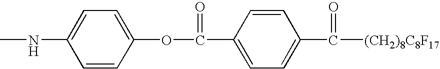 |
| C-12 | 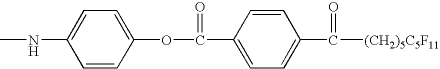 |
| C-13 | 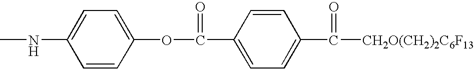 |
| C-14 | 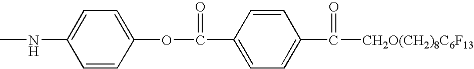 |
| C-15 | 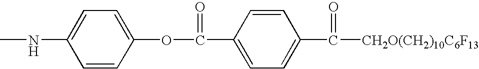 |
| C-16 | 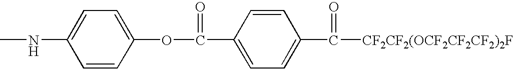 |
| C-17 | 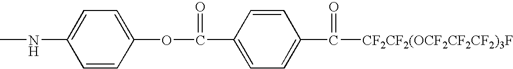 |
| C-18 | 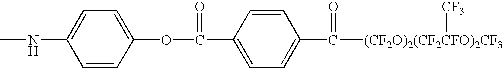 |
| C-19 | 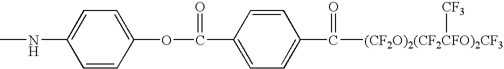 |
| C-20 | 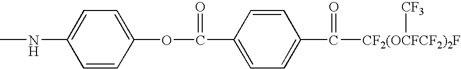 |
| E-1 | 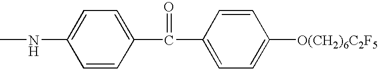 |
| E-2 | 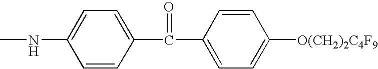 |
| E-3 | 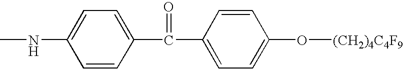 |
| E-4 | 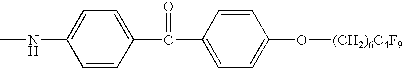 |
| E-5 | 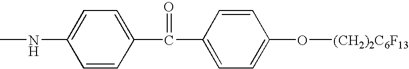 |
| E-6 | 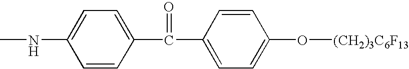 |
| E-7 | 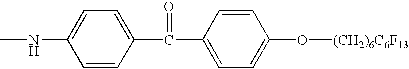 |
| E-8 | 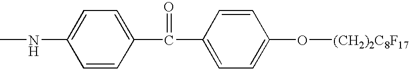 |
| E-9 | 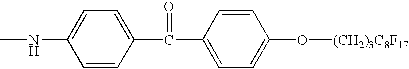 |
| E-10 | 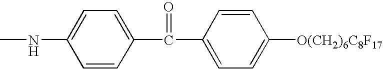 |
| E-11 | 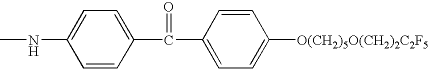 |
| E-12 | 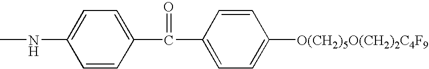 |
| E-13 | 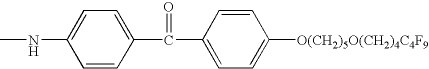 |
| E-14 | 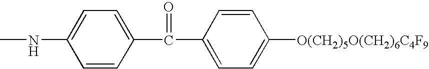 |
| E-15 | 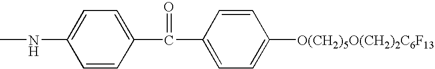 |
| E-16 | 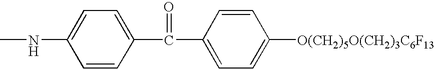 |
| E-17 | 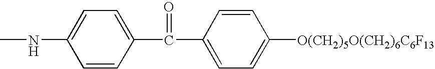 |
| E-18 | 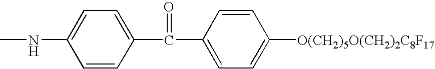 |
| E-19 | 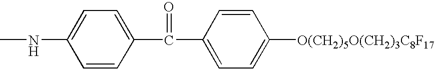 |
| E-20 | 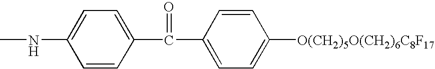 |
| E-21 | 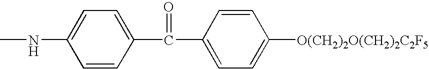 |
| E-22 | 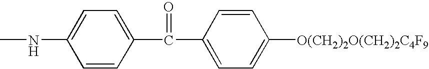 |
| E-23 | 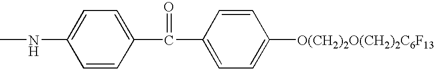 |
| E-24 | 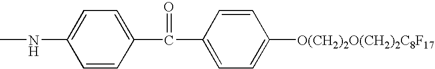 |
| E-25 | 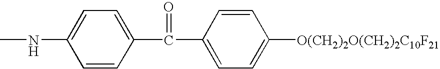 |
| F-1 | 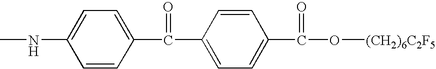 |
| F-2 | 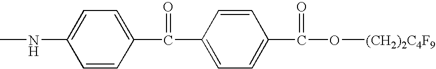 |
| F-3 | 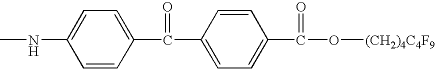 |
| F-4 | 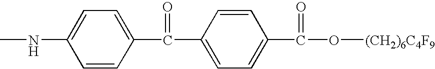 |
| F-5 | 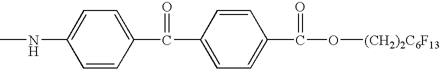 |
| F-6 | 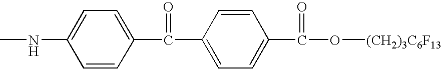 |
| F-7 | 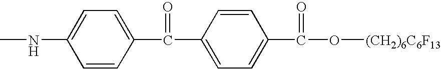 |
| F-8 | 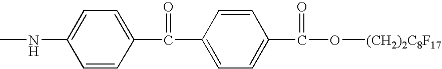 |
| F-9 | 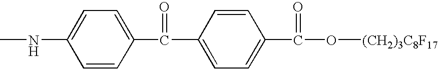 |
| F-10 | 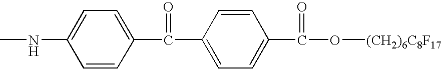 |
| F-11 | 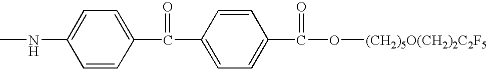 |
| F-12 | 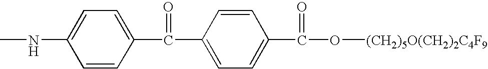 |
| F-13 | 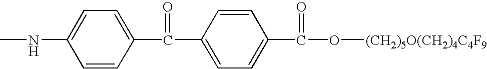 |
| F-14 | 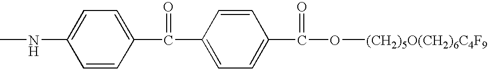 |
| F-15 | 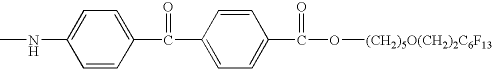 |
| F-16 | 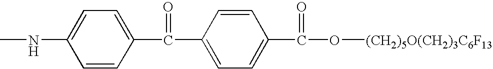 |
| F-17 | 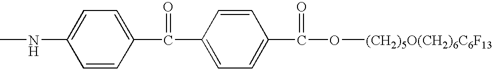 |
| F-18 | 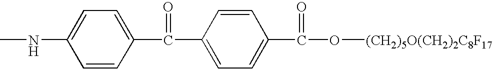 |
| F-19 | 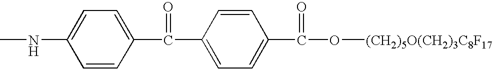 |
| F-20 | 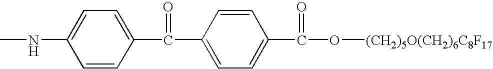 |
| F-21 | 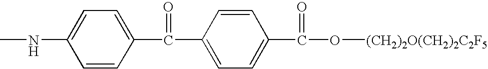 |
| F-22 | 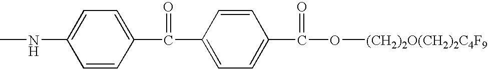 |
| F-23 | 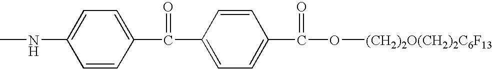 |
| F-24 | 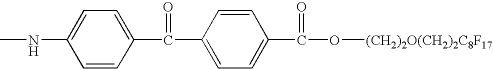 |
| F-25 | 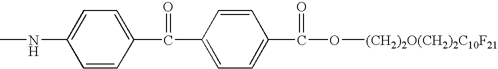 |
| F-26 | 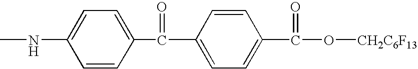 |
| F-27 | 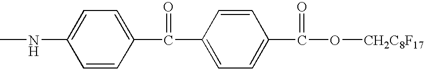 |
| F-28 | 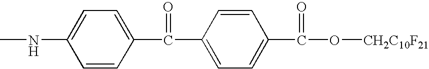 |
| F-29 | 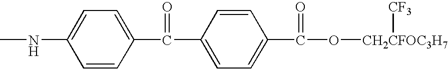 |
| F-30 | 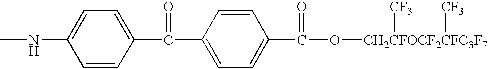 |
| F-31 | 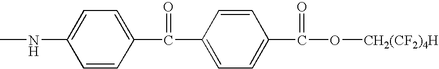 |
| F-32 | 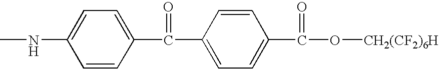 |
| F-33 | 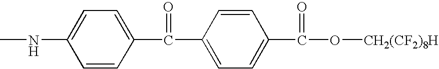 |
| F-34 | 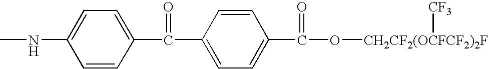 |
| F-35 | 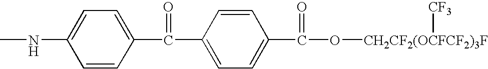 |
| F-36 | 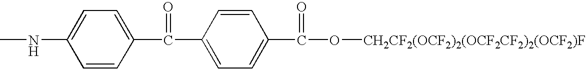 |
| F-37 | 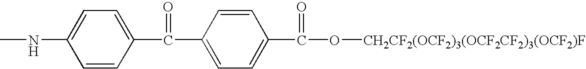 |
| F-38 | 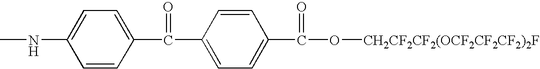 |
| F-39 | 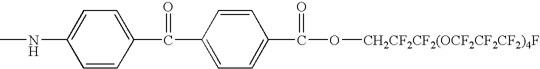 |
| F-40 | 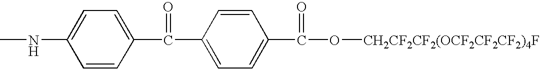 |
| G-1 | 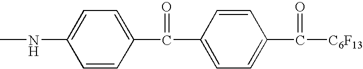 |
| G-2 | 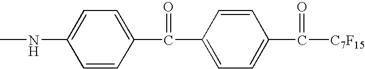 |
| G-3 | 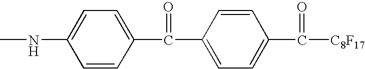 |
| G-4 | 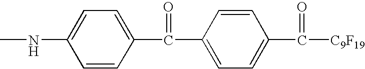 |
| G-5 | 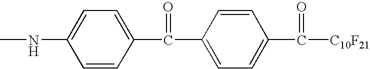 |
| G-6 | 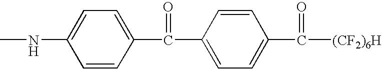 |
| G-7 | 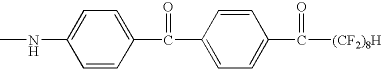 |
| G-8 | 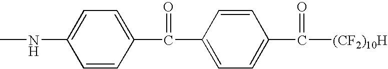 |
| G-9 | 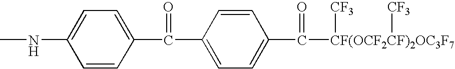 |
| G-10 | 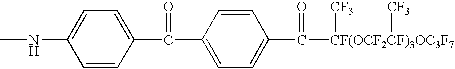 |
| G-11 | 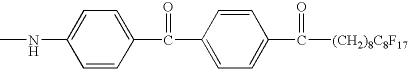 |
| G-12 | 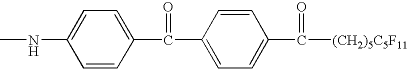 |
| G-13 | 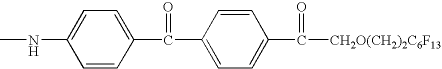 |
| G-14 | 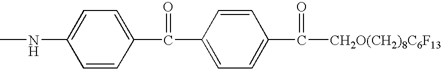 |
| G-15 | 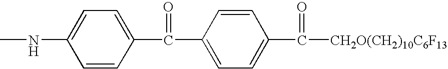 |
| G-16 | 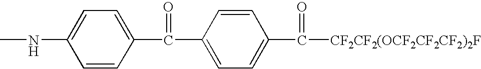 |
| G-17 | 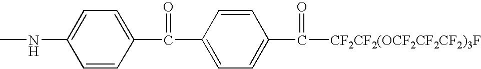 |
| G-18 | 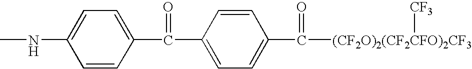 |
| G-19 | 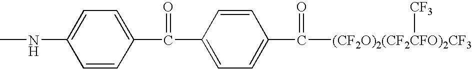 |
| G-20 | 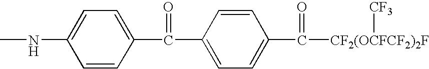 |
| T-1 | 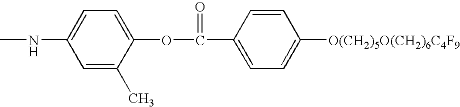 |
| T-2 | 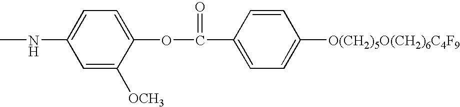 |
| T-3 | 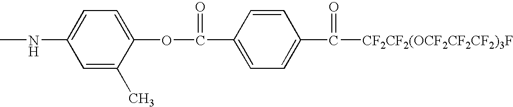 |
| T-4 | 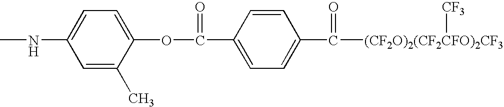 |
| T-5 | 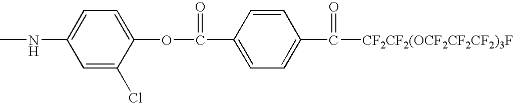 |
| T-6 | 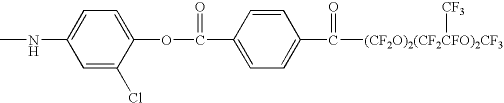 |
| T-7 | 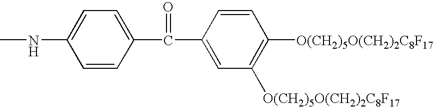 |
| T-8 | 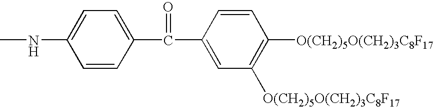 |
| T-9 | 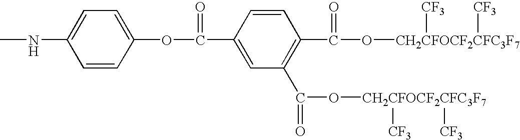 |
| T-10 | 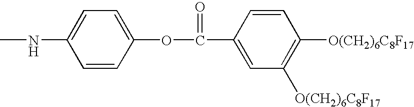 |

Examples of the method of producing the compound of the invention include a method which comprises reacting a disc-shaped compound which is a stereoparent of triazine ring with one or more side moieties (e.g., nucleophilic substitution reaction or coupling reaction of cyanuric chloride) and a method which comprises constructing a cyclic structure from one or more compounds comprising the side moiety to produce a triazine ring compound. Preferred among these production methods is a synthesis method involving the reaction of cyanuric chloride with a compound having active hydrogen (e.g., derivatives such as amine, aniline, alcohol, phenol, thioalcohol and thiophenol). For details, reference can be made to synthesis examples described later.

Examples of the organic solvent to be used in the reaction include halogenated hydrocarbon-based organic solvents (e.g., dichloromethane), ester-based organic solvents (e.g., methyl acetate, ethyl acetate), ketone-based organic solvents (e.g., acetone, methyl ethyl ketone), ether-based organic solvents (e.g., tetrahydrofurane, dioxane), nitrile-based organic solvents (e.g., acetonitrile, propionitrile), amide-based organic solvents (e.g., N,N-dimethylformamide, N,N-dimethylacetamide, 1,3-dimethyl-2-imidazolidone, 1,3-dimethyl-3,4,5,6-tetrahydro-2(1H)-pyrimidinone (DMPU), triamide hexamethylphosphate), and sulfoxide-based organic solvents (e.g., dimethyl sulfoxide). The reaction may be effected in the presence of a catalyst and a base as necessary.

The compound represented by the formula (1) can be used as a base oil of lubricant composition per se. In general, however, the compound of the formula (1) is used as a base oil of the lubricant composition of the invention in admixture with a mineral oil or synthetic oil to be used as a base oil of lubricant composition. The mineral oil or synthetic oil to be used as a mixed lubricant base oil is not specifically limited. As the mineral oil or synthetic oil there may be used any mineral oil or synthetic oil that is commonly used as a lubricant base oil. Examples of the material corresponding to such a lubricant base oil include mineral oil, synthetic oil, and mixture thereof. Examples of the mineral oil employable herein include solvent-purified raffinate obtained by processing a lubricant raw material derived by distillation of a paraffinic, intermediate group-based or naphthenic crude oil under normal or reduced pressure with an aromatic extracting solvent such as phenol, furfural and N-methylpyrrolidone, hydrogenated oil obtained by bringing a lubricant raw material into contact with hydrogen in the presence of a hydrogenation catalyst such as cobalt and molybdenum supported on silica-alumina under hydrogenation conditions, hydrogenated cracked petroleum obtained by bringing a lubricant raw material into contact with hydrogen in the presence of a hydrogenation decomposition catalyst under severe decomposition reaction conditions, isomerized oil obtained by bringing a wax into contact with hydrogen in the presence of an isomerization catalyst under isomerization conditions, and lubricant fraction obtained by a solvent purifying step in combination with a hydrogenation step, hydrogenation decomposition step, isomerization step, etc. In particular, a high viscosity mineral oil obtained by a hydrogenation decomposition step or isomerization step is preferred. In any of these production methods, a step such as dewaxing step, hydrogenation finishing step and clay treatment step may be any ordinary step. Specific examples of the mineral oil employable herein include light-weight neutral oil, middle-weight neutral oil, heavy-weight neutral oil, and bright stock. These mineral oils may be properly mixed so as to satisfy the requirements to prepare a desired base oil. Examples of the synthetic oil employable herein include poly-α-olefin, α-olefin oligomer, polybutene, alkyl benzene, polyol ester, dibasic acid ester, polyoxyalkylene glycol, polyoxyalkylene glycol ether, and silicone oil. These base oils may be used singly or in combination of two or more thereof. Alternatively, the mineral oil and the synthetic oil may be used in combination. The mixture of mineral oil and synthetic oil may be used as a mixed base oil of lubricant composition of the invention. Such an ordinary base oil normally exhibits a dynamic viscosity of from 2 to 20 mm²/s, preferably from 3 to 15 mm²/s at 100° C. A mixed base oil having an optimum dynamic viscosity is properly selected to comply with the lubricating conditions at the mechanical frictional sliding portion to which the lubricant composition of the invention is applied.

Referring to the mixing proportion of the compound represented by the formula (1) and the ordinary base oil in the lubricant composition of the invention, the mixing proportion of the compound represented by the formula (1) is normally from 0.1 to 20% by mass and the mixing proportion of the ordinary base oil, i.e., mineral oil and/or synthetic oil is from 80% to 99.9% by mass based on the total amount of the base material oil. Preferably, the mixing proportion of the compound represented by the formula (1) is from 0.1% to 10% by mass, most preferably from 0.1% to 5% by mass. As previously mentioned, however, the compound comprising the compound of the invention can be used as a base oil of lubricant composition per se. In many cases, this compound can be used singly to advantage. The use of this compound makes it possible to provide a low friction coefficient as well as an excellent abrasion resistance within a wide temperature range even under severe lubricating conditions.

The lubricant composition of the invention comprises the compound represented by the formula (1) incorporated therein as a main component. In order to secure practical performance adapted for various purposes, the lubricant composition of the invention may comprise various additives for lubricant such as bearing oil, gear oil and power transmission oil, e.g., abrasion inhibitor, extreme pressure agent, oxidation inhibitor, viscosity index improver, detergent-dispersant, metal deactivator, corrosion inhibitor, rust preventive, antifoaming agent incorporated therein in a proper amount so far as the aim of the invention cannot be impaired.

The lubricant composition of the invention is characterized in that it exhibits a low friction coefficient and excellent abrasion resistance and extreme pressure properties under severe lubricating conditions. The lubricant composition of the invention can comprise a compound having a structure represented by the formula (1), preferably the formula (2), more preferably the formula (3), (4) or (5), incorporated therein in a variety of selections so that it is adapted for desired purposes or working atmospheres. For example, a practical lubricant composition which can be used even within a wide temperature range can be provided.

In accordance with the invention, the aforementioned characteristics can be made the use of, to provide an excellent lubricant composition which can exhibit a high abrasion resistance and a low friction coefficient without causing seizing under severe lubricating conditions on which the related art lubricants or greases can no longer supply oil film and thus allows energy saving when applied to bearing or gear under severe lubricating conditions. The invention also provides a lubricant composition which causes no seizing under severe lubricating conditions, making it possible to enhance the reliability of sliding units and contribute to the reduction of size of sliding units.

The invention will be further described hereinafter in the following examples. The materials, reagents, their mixing proportions, procedures, etc. may be properly changed without departing from the scope of the invention. Accordingly, the scope of the invention should not be limited to the following examples.

### SYNTHESIS EXAMPLE

### Synthesis of E-9

An inventive compound E-9 was synthesized according to the following reaction scheme.

(Synthesis of 4,4,5,5,6,6,7,7,8,8,9,9,9tridecafluorononyl-oxybenzene)

Into a 500 ml reaction vessel equipped with an agitator, a reflux condenser and a thermometer were chaerged 45.4 g (0.12 mol) of 4,4,5,5,6,6,7,7,8,8,9,9,9-tridecafluorononyl alcohol, 13.2 g (0.13 mol) of triethylamine and 150 ml of tetrahydrofurane. These components were then stirred to obtain a solution. The solution was then cooled to 0° C. To the solution was then added dropwise a tetrahydrofurane solution of 13.7 g (0.12 mol) of methanesulfonyl chloride. The mixture was gradually heated to room temperature where it was then stirred for 2 hours. The mixture was then extracted with ethyl acetate. The resulting organic phase was washed with water, dispensed, and then dried over anhydrous magnesium sulfate. The solvent was then distilled off under reduced pressure to obtain a crude reaction product.

Into a 500 ml reaction vessel equipped with an agitator, a reflux condenser and a thermometer were charged 9.4 g (0.1 mol) of phenol, 50.1 g of (0.11 mol) of the methanesulfonyl derivative obtained above and 150 mol of N,N-dimethylformamide. These components were then stirred to obtain a solution. To the solution was then added 16.6 g (0.12 mol) of potassium carbonate. The mixture was heated to 98° C. where it was then stirred for 2 hours. The mixture was allowed to cool to room temperature where it was then extracted with ethyl acetate. The resulting organic phase was washed with water, dispensed, and then dried over anhydrous magnesium sulfate. The solvent was then distilled off under reduced pressure to obtain a crude reaction product. The crude reaction product was then subjected to purification by silica gel column dispensation to obtain 43.6 g (96%) of the desired compound.

(Synthesis of Nitro Derivative)

Into a 300 ml reaction vessel equipped with an agitator, a dropping funnel and a thermometer were charged 100 ml of methylene chloride and 13.3 g (0.1 mol) of aluminum chloride. These components were then stirred to obtain a solution. The solution was then cooled to 0° C. To the solution was then added dropwise a methylene chloride solution of 16.7 g (0.09 mol) of nitrobenzoyl chloride. The mixture was then stirred for 30 minutes. To the mixture was then added dropwise a methylene chloride solution of 40.9 g (0.09 mol) of the alkoxybenzene derivative obtained above in 1 hour. After the dropwise addition, the mixture was stirred at room temperature for 1.5 hours. The reaction solution was poured into ice-water which was then acidified with hydrochloric acid. The reaction solution was then extracted with ethyl acetate. The resulting organic phase was washed with water, dispensed, and then dried over anhydrous magnesium sulfate. The solvent was distilled off under reduced pressure to obtain a crude reaction product. The crude reaction product was then purified with methanol to obtain 50.0 g (83%) of the desired compound.

(Synthesis of Aniline Derivative)

Into a 1 l reaction vessel equipped with an agitator, a reflux condenser and a thermometer were charged 22.3 g (0.4 mol) of reduced iron, 220 ml of isopropyl alcohol, 22 ml of water and 2.1 g of ammonium chloride. These components were then heated to 90° C. with stirring under reflux. To the mixture was then added gradually 48.2 g (0.08 mol) of the nitro compound obtained above. After the addition, the mixture was then heated with stirring for 2 hours. After the reaction, the mixture was subjected to celite filtration while being kept heated, and then washed with ethyl acetate. The filtrate was extracted with ethyl acetate, and then washed with water. The resulting organic phase was dispensed, and then dried over anhydrous magnesium sulfate. The solvent was distilled off under reduced pressure to obtain a crude reaction product. The crude reaction product was then subjected to purification by silica gel column dispensation to obtain 56.88 g (99%) of the desired compound.

(Synthesis of Compound E-9)

Into a 300 ml reaction vessel equipped with an agitator, a reflux condenser and a thermometer were charged 100 ml of methyl ethyl ketone and 3.68 g (0.02 mol) of cyanuric chloride. These components were then stirred to obtain a solution. The solution was then cooled to 0° C. To the solution was then added dropwise slowly a methyl ethyl ketone solution of 41.3 g (0.072 mol) of the aniline derivative compound obtained above. Subsequently, to the mixture was added 11 g (0.08 mol) of potassium carbonate. The mixture was stirred at room temperature for 30 minutes, and then heated to 98° C. with stirring for 2 hours. The reaction product was allowed to cool to room temperature, and then extracted with ethyl acetate. The resulting organic phase was then washed with water. The organic phase was dispensed, and then dried over anhydrous magnesium sulfate. The solvent was then distilled off under reduced pressure to obtain a crude reaction product. The crude reaction product was then purified with methanol to obtain 25.1 g (yield: 70%) of the desired compound (E-9).

NMR data of the compound thus synthesized will be given below.

¹HNMR (300 MHz, CDCl₃): δ7.59 (m, 6H), 7.45 (m, 6H), 6.87 (m, 6H), 6.56 (m, 6H), 3.94 (t, 6H), 1.71 (m, 6H), 1.61 (t, 6H)

### EXAMPLES 1 TO 6 AND COMPARATIVE EXAMPLES 1 TO 6

### Evaluation of Properties of Lubricant Composition

The inventive compounds A-9, B-18, C-9, E-25, F-7 and G-13, lubricant base oils and the following comparative compounds M-1 and M-2 were subjected to friction test under the following conditions to measure the friction coefficient thereof. For the measurement of friction coefficient in these examples, the samples were subjected to friction test using a reciprocating friction testing machine (SRV friction abrasion testing machine) under the following testing conditions. The results of Examples 1 to 6 are set forth in Table 1. Similarly, the results of Comparative Examples 1 to 6 are set forth in Table 1.

(Testing Conditions)

The friction test was effected using a cylinder-on-plate system.

Test specimen (friction material): SUJ-2

Plate: φ24×6.9 mm

Cylinder: φ11×15 mm

Temperature: 220° C.

Load: 400 N

Amplitude: 1.5 mm

Frequency: 50 Hz

Testing time: Measured 30 minutes and 10 hours after the beginning of test

TABLE 1

| | | Friction | |
|---|---|---|---|
| | | coefficient | |
| | | after | Friction coefficient after |
| | Compound No. | 30 min. | 10 hr. |
| Example 1 | Inventive compound A-9 | 0.034 | 0.033 |
| Example 2 | Inventive compound B-18 | 0.031 | 0.031 |
| Example 3 | Inventive compound C-9 | 0.033 | 0.033 |
| Example 4 | Inventive compound E-25 | 0.03 | 0.03 |
| Example 5 | Inventive compound F-7 | 0.03 | 0.03 |
| Example 6 | Inventive compound G-13 | 0.03 | 0.03 |
| Comparative Example 1 | Pentaerythritol ester | 0.25 | Test stopped at 0.3 or more |
| Comparative Example 2 | Alkylbenzene | 0.25 | Test stopped at 0.3 or more |
| Comparative Example 3 | Naphthenic mineral oil | 0.27 | Test stopped at 0.3 or more |
| Comparative Example 4 | Paraffinic mineral oil | 0.27 | Test stopped at 0.3 or more |
| Comparative Example 5 | Comparative compound M-1 | 0.18 | Test stopped at 0.3 or more |
| Comparative Example 6 | Comparative compound M-2 | 0.18 | Test stopped at 0.3 or more |

Comparative Compound (M-1)
[Different from Exemplary Compound LUB-20 Disclosed in JP-A-2002-69472 only in the Number of Carbon Atoms in the Alkoxy Group] 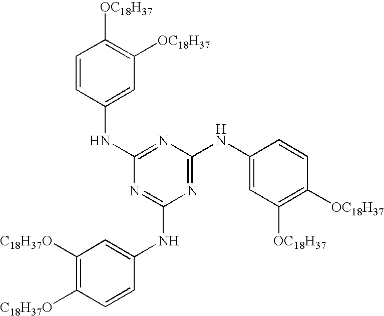
Comparative Compound (M-2)
[Same as Exemplary Compound LUB-28 Disclosed in JP-A-2002-69472]

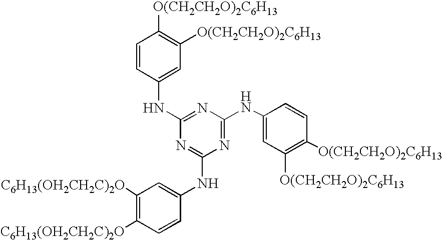

As can be seen in Table 1, the compounds of the invention exhibited a considerably low friction coefficient after 30 minutes and 10 hours as compared with the lubricant base oils and the comparative compounds.

The lubricant composition of the invention exhibits an excellent effect of giving a high abrasion resistance, high extreme pressure properties and a low friction at mechanical frictional sliding portion and has a high practicality. The lubricant composition of the invention also exhibits excellent lubricating properties and maintains a low friction over an extended period of time even within a high temperature range and thus has a high industrial applicability.

Further, the compound of the invention exhibits excellent lubricating properties not only when used in admixture with a lubricant base oil but also when used free of lubricant base oil. The lubricant comprising the compound of the invention incorporated therein has the properties of the lubricant composition of the invention and thus has a high industrial applicability.

The present disclosure relates to the subject matter contained in Japanese Patent Application No. 279100/2004 filed on Sep. 27, 2004, which is expressly incorporated herein by reference in its entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims set forth below.

What is claimed is:

1. A lubricant composition comprising a compound represented by the following formula (1): 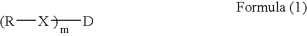
wherein D represents a cyclic group which can bond to m groups of R—X—'s; X's each independently represent a single bond, an NR¹ group, an oxygen atom, a sulfur atom, a carbonyl group, a sulfonyl group or a divalent linking group comprising these groups in combination; R¹ represents a hydrogen atom or a C₁-C₃₀ alkyl group; R's each independently represent an alkyl group, an alkenyl group, an alkynyl group, an aryl group or a heterocyclic group; at least one of m groups of R's contains a partly fluorinated hydrocarbon group or a fluorinated carbon group; and m represents an integer of from 2 to 11.

2. The lubricant composition as defined in claim 1, wherein D is a 5- to 7-membered heterocyclic residue.

3. The lubricant composition as defined in claim 2, wherein the formula (1) is represented by the following formula (2):
Formula (2) 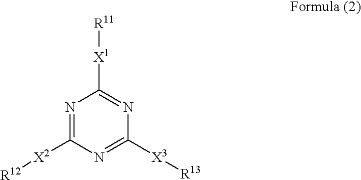
wherein X¹, X² and X³ each independently represent a single bond, an NR¹ group, an oxygen atom, a sulfur atom, a carbonyl group, a sulfonyl group or a divalent linking group comprising these groups in combination; R¹ represents a hydrogen atom or a C₁-C₃₀ alkyl group; and R¹¹, R¹² and R¹³ each independently represent a substituted or unsubstituted alkyl, alkenyl, alkynyl, aryl or heterocyclic group, with the proviso that at least one of R¹¹, R¹² and R¹³ contains a partly fluorinated hydrocarbon group or a fluorinated carbon group.

4. The lubricant composition as defined in claim 3, wherein the formula (1) is represented by the following formula (3): 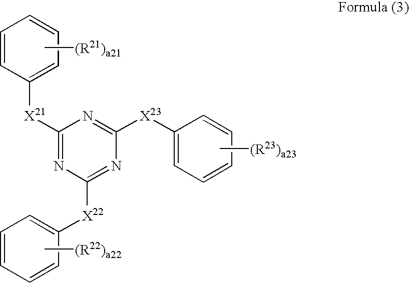
wherein X²¹, X²² and X²³ each independently represent a single bond, an NR¹ group, an oxygen atom, a sulfur atom, a carbonyl group, a sulfonyl group or a divalent linking group comprising these groups in combination; R¹ represents a hydrogen atom or a C₁-C₃₀ alkyl group; R²¹, R²² and R²³ each independently represent a substituent, with the proviso that at least one of R²¹, R²² and R²³ contains a partly fluorinated hydrocarbon group or a fluorinated carbon group; and a21, a22 and a23 each independently represent an integer of from 1 to 5.

5. The lubricant composition as defined in claim 4, wherein the formula (1) is represented by the following formula (4): 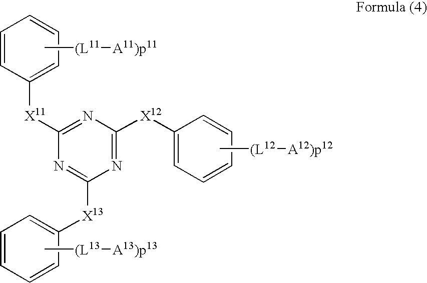
wherein X¹¹, X¹² and X¹³ each independently represent a single bond, an NR¹ group, an oxygen atom, a sulfur atom, a carbonyl group, a sulfonyl group or a divalent linking group comprising these groups in combination; L¹¹, L¹² and L¹³ each independently represent a single bond, an NR¹ group, an alkylene group, an oxygen atom, a sulfur atom, a carbonyl group, a sulfonyl group or a divalent linking group comprising these groups in combination; R¹ is a hydrogen atom or a C₁-C₃₀ alkyl group; A¹¹, A¹² and A¹³ each independently represent an aromatic group or a heterocyclic group, with the proviso that at least one of A¹¹, A¹² and A¹³ contains a partly fluorinated hydrocarbon group or a fluorinated carbon group; and p¹¹, p¹² and p¹³ each independently represent an integer of from 1 to 5.

6. The lubricant composition as defined in claim 5, wherein all of X¹¹, X¹² and X¹³ in the formula (4) are imino group (—NH—).

7. The lubricant composition as defined in claim 6, wherein the formula (4) is represented by the following formula (5): 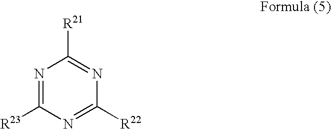
wherein R²¹, R²² and R²³ each independently are represented by the following formula (6): 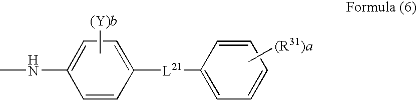
wherein L²¹ represents a single bond, an NR¹ group, an alkylene group, an oxygen atom, a sulfur atom, a carbonyl group, a sulfonyl group or a divalent linking group comprising these groups in combination; R¹ is a hydrogen atom or a C₁-C₃₀ alkyl group; R³¹ represents a substituent, with the proviso that at least one of a groups of R³¹'s contains a partly fluorinated hydrocarbon group or a fluorinated carbon group; the suffix a represents an integer of from 1 to 5; Y represents a substituent; and the suffix b represents 0 or an integer of from 1 to 4.

8. A triazine compound represented by the following formula (2): 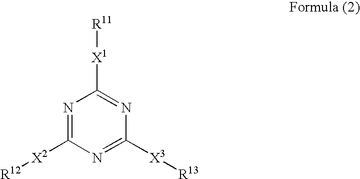
wherein X¹, X² and X³ each independently represent a single bond, an NR¹ group, an oxygen atom, a sulfur atom, a carbonyl group, a sulfonyl group or a divalent linking group comprising these groups in combination; R¹ is a hydrogen atom or a C₁-C₃₀ alkyl group; and R¹¹, R¹² and R¹³ each independently represent a substituted or unsubstituted alkyl, alkenyl, alkynyl, aryl or heterocyclic group, with the proviso that at least one of R¹¹, R¹² and R¹³ contains a partly fluorinated hydrocarbon group or a fluorinated carbon group.

9. The triazine compound as defined in claim 8, wherein the formula (2) is represented by the following formula (3): 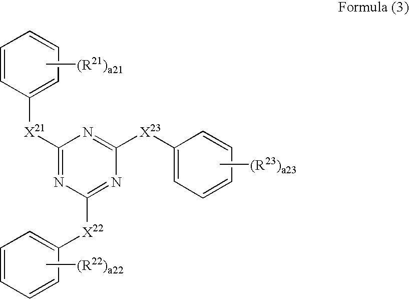
wherein X²¹, X²² and X²³ each independently represent a single bond, an NR¹ group, an oxygen atom, a sulfur atom, a carbonyl group, a sulfonyl group or a divalent linking group comprising these groups in combination; R¹ is a hydrogen atom or a C₁-C₃₀ alkyl group; R²¹, R²² and R²³ each independently represent a substituent, with the proviso that at least one of R²¹, R²² and R²³ contains a partly fluorinated hydrocarbon group or a fluorinated carbon group; and a21, a22 and a23 each independently represent an integer of from 1 to 5.

10. A triazine compound represented by the following formula (4): 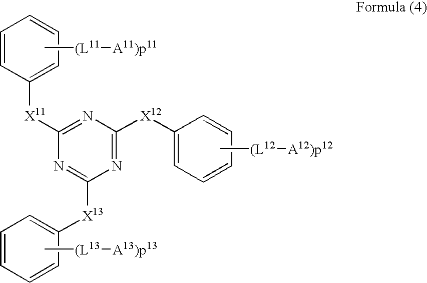
wherein X¹¹, X¹² and X¹³ each independently represent a single bond, an NR¹ group, an oxygen atom, a sulfur atom, a carbonyl group, a sulfonyl group or a divalent linking group comprising these groups in combination; L¹¹, L¹² and L¹³ each independently represent a single bond, an NR¹ group, an alkylene group, an oxygen atom, a sulfur atom, a carbonyl group, a sulfonyl group or a divalent linking group comprising these groups in combination; R¹ is a hydrogen atom or a C₁-C₃₀ alkyl group; A¹¹, A¹² and A¹³ each independently represent an aromatic group or a heterocyclic group, with the proviso that at least one of A¹¹, A¹² and A¹³ contains a partly fluorinated hydrocarbon group or a fluorinated carbon group; and p¹¹, p¹² and p¹³ each independently represent an integer of from 1 to 5.

11. The triazine compound as defined in claim 10, wherein all of X¹¹, X¹² and X¹³ in the formula (1) are imino group (—NH—).

12. The triazine compound, wherein the formula (4) is represented by the following formula (5): 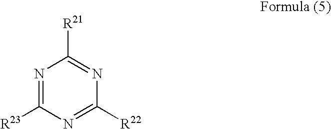
wherein R²¹, R²² and R²³ each independently are represented by the following formula (6): 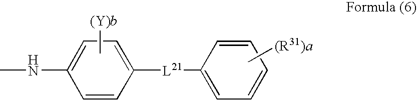
wherein L²¹ represents a single bond, an NR¹ group, an alkylene group, an oxygen atom, a sulfur atom, a carbonyl group, a sulfonyl group or a divalent linking group comprising these groups in combination; R¹ is a hydrogen atom or a C₁-C₃₀ alkyl group; R³¹ represents a substituent, with the proviso that at least one of a groups of R³¹'s contains a partly fluorinated hydrocarbon group or a fluorinated carbon group; the suffix a represents an integer of from 1 to 5; Y represents a substituent; and the suffix b represents 0 or an integer of from 1 to 4.
